(19) United States
(12) Patent Application Publication
(10) Pub. No.: US 2025/0325493 A1
(43) Pub. Date: Oct. 23, 2025

(54) COMPOSITIONS AND METHODS FOR MAKING AND USING POLYMER-COATED NANOCAPSULES FOR TARGETED PHARMACEUTICAL AGENT DELIVERY

(71) Applicants: The Regents of the University of Colorado, a body Corporate, Denver, CO (US); Colorado School of Mines, Golden, CO (US)

(72) Inventors: Nikki Farnsworth, Golden, CO (US); Holger Russ, Gainesville, FL (US); Jillian Collins, Golden, CO (US)

(21) Appl. No.: 19/197,525

(22) Filed: May 2, 2025

Related U.S. Application Data
(63) Continuation of application No. PCT/US23/81402, filed on Nov. 28, 2023.
(60) Provisional application No. 63/428,319, filed on Nov. 28, 2022.

(51) Int. Cl. A61K 9/51; A61K 45/06

(52) U.S. Cl. CPC A61K 9/5123; A61K 9/5146; A61K 9/5161; A61K 9/5169; A61K 45/06

(57) ABSTRACT

Embodiments of the present disclosure provide novel compositions and methods for making and using polymer-coated nanocapsules. In certain embodiments, compositions and methods are disclosed for embedding at least one agent in a liquid fatty acid composition to form an inner core of the polymer-coated nanocapsule and coating the at least one agent-containing liquid fatty acid composition inner core with polymer to form at least one coating layer of polymer that further includes at least one positively charged surfactant (e.g., cationic surfactant), forming polymer-coated nanocapsules. In certain embodiments, the at least one positively charged surfactant binds to at least one targeting agent for directed use of the polymer-coated nanocapsules.

### CROSS-REFERENCE TO RELATED APPLICATIONS

This U.S. Continuation Application claims priority to International Application No. PCT/US2023/081402, filed Nov. 28, 2023, which claims priority to U.S. Provisional Application No. 63/428,319, filed Nov. 28, 2022. These applications are incorporated herein by reference in their entireties for all purposes.

### GOVERNMENT SUPPORT

This invention was made with government support under pilot grant funding through P30DK11607 awarded by National Institutes of Health and funding through 1-INO-2022-1231-S-B and 3-SRA-2023-1367-S-B awarded by the Juvenile Diabetes Research Foundation (JDRF). The government has certain rights in the invention.

### FIELD

Embodiments of the present disclosure provide novel compositions and methods for making and using polymer-coated nanocapsules. In certain embodiments, compositions and methods are disclosed for embedding at least one agent in a liquid fatty acid composition to form an inner core of the nanocapsule and coating the at least one agent-containing liquid fatty acid composition inner core with a polymer to form at least one coating layer of polymer that includes at least one positively charged surfactant (e.g., cationic surfactant), forming polymer-coated nanocapsules. In certain embodiments, the at least one positively charged surfactant binds to at least one targeting agent for directed targeting and use of the polymer-coated nanocapsules.

### BACKGROUND

Therapeutic impacts of therapeutic agents can be compromised by many challenges. One challenge is targeted delivery of a therapeutic agents to a particular site of a subject. For example, targeted delivery of therapeutic agents to a particular cell type or organ of a subject can be difficult with respect to targeting the site and delivering enough of the therapeutic agent to the site. In addition, therapeutic agents often exhibit increased instability during delivery within a subject to reach a targeted site and can cause significant side effects if delivered to the wrong site or require significantly more therapeutic agent to accomplish a desired outcome due to loss of the therapeutic agent during the process of administration and delivery. Therefore, there is a need for improved methods for targeted therapeutic agent delivery to reduce loss of agent and reduce side effects, for example.

### SUMMARY

Embodiments of the present disclosure provide novel compositions and methods for making and using polymer-coated nanocapsules. In certain embodiments, compositions and methods are disclosed for embedding at least one therapeutic agent in a composition including at least one fatty acid to form an inner core; at least one layer of a shell coating the inner core, the coating layer including at least one biodegradable polymer to make a biodegradable polymer shell coating where the at least one coating layer of the at least one biodegradable polymer shell coating further includes at least one positively charged surfactant on the surface of the at least one biodegradable polymer shell coating layer(s); and at least one targeting agent associated with at least one of the at least one positively charged surfactant. In certain embodiments, the at least one therapeutic agent can include at least one hydrophilic or hydrophobic therapeutic agent. In some embodiments, the at least one hydrophilic or hydrophobic therapeutic agent can be encased in formulations designed for addressing these different properties.

In some embodiments as indicated in paragraph above, the at least one biodegradable polymer includes at least one of polycaprolactone (PCL), poly(lactic-co-glycolic acid) (PLGA), polylactic acid (PLA), chitosan, gelatin, and a biodegradable polymer monomer size ranging from about 0.1 kDa to about 80 kDa. In some embodiments, the at least one biodegradable polymer includes at least polycaprolactone (PCL). In other embodiments, the at least one positively charged surfactant on the surface of the at least one biodegradable polymer shell coating layer further includes at least one negatively charged agent, where the at least one negatively charged agent is capable of associating with or linking to the at least one positively charged surfactant and the at least one targeting agent. In accordance with these embodiments, the at least one negatively charged agent can include at least one of hyaluronic acid, aggrecan, versican, syndecan, nidogen, decorin, biglycan, chondroitin sulfate, keratin sulfate, γ-polyglutamic acid, oligocthylene glycol, or other negatively charged protein or other brush polymer.

In other embodiments, the nanocapsule according to any of the preceding paragraphs can include an inner core of at least one liquid fatty acid composition made up of at least one liquid lipid composition. In certain embodiments, the at least one liquid fatty acid is made up of at least one naturally-occurring biocompatible liquid fatty acid. In certain embodiments, the at least one naturally-occurring biocompatible liquid fatty acid includes a fatty acid that is liquid at room temperature (e.g., 20-25° C.)

In certain embodiments and further to paragraphs [0005]-[0007] above, the at least one liquid fatty acid that makes up part of the inner core includes at least one of: coconut oil, sunflower oil, vegetable oil, soybean oil, colza oil, peanut oil, mineral oil, corn oil, olive oil, palm oil, cottonseed oil, castor oil, linseed oil, borage oil, evening primrose oil, marine oils, fish oils, algae oils, oils derived from petroleum, liquid paraffin, short-chain fatty alcohols, medium-chain aliphatic branched fatty alcohols, fatty acid esters with short-chain alcohols, isopropyl myristate, isopropyl palmitate, medium-chain triglycerides, capric and caprylic triglycerides, and mixtures thereof. In some embodiments, the at least one fatty acid includes at least coconut oil or the like.

In certain embodiments and further to paragraphs [0005]-[0008] above, the at least one targeting agent can include at least one of a polypeptide, a polynucleotide, a chimeric molecule, a glycoprotein, a whole organism, a whole cell, a pathogen, a toxin, a polysaccharide, a small molecule, an immunoglobulin (e.g., antibody, monoclonal antibody), a fragment or segment thereof, a metabolite, a chemical classified as a chemical or biological agent or other pharmaceutical agent thereof. In some embodiments, the at least one targeting agent can include at least one of a polypeptide, a polynucleotide, a chimeric molecule, a glycoprotein, an immunoglobulin where the at least one targeting agent targets a receptor or other marker on a target of interest (e.g., a cell or tissue, etc.)

In some embodiments and further to paragraphs [0005]-[0009] above, the at least one targeting agent can include at least one of an antibody, a ligand, a receptor, an enzyme, a viral antigen, a bacterial antigen, a yeast antigen, a toxin, a recombinant peptide, a recombinant protein, a polypeptide derived from a target protein or pathogen, a synthetic peptide or protein, a polynucleotide derived from a target protein or pathogen, a virus-like particle, a live virus, a live, attenuated virus, an inactivated virus. In certain embodiments and further to paragraphs [0005]-[0009] above, the at least one targeting agent includes at least one of a polypeptide or an antibody. In certain embodiments, the at least one targeting agent that includes at least one of a polypeptide or an antibody includes a polypeptide or an antibody capable of associating with a specific molecule associated with a target cell population (e.g., pancreatic b cells, kidney cells, cardiac cells, liver cells, lung cells), a targeted organ, or a targeted region (e.g., gastrointestinal tract) in a subject (e.g., glucagon-like peptide-1 receptor (GLP-1R) agonist Exendin-4; antibody against a specific b-cell marker ENTPD3 (NTPDase3)), of a targeted cell population (e.g., pancreatic b cells). In accordance with these embodiments, the at least one targeting agent directs the polymer coated nanocapsules containing at least one therapeutic agent to the target for the at least one polymer coated nanocapsules containing at least one therapeutic agent to be engulfed (e.g., by a targeted cell), release the at least one therapeutic agent and further treat, prevent onset or reduce risk of a health condition.

In certain embodiments and further to paragraphs [0005]-[0010] above, the at least one targeting agent includes at least one small molecule or at least one metabolite and the at least one small molecule or the at least one metabolite includes a low molecular weight organic compound. In accordance with these embodiments, the at least one small molecule or the at least one metabolite includes at least one of a fatty acid, glucose, an amino acid, cholesterol, a lipid, glycoside, alkaloid, and natural phenols capable of targeting a cell, tissue, or other targeted region in order to direct engulfment of the polymer-coated nanocapsules.

In other embodiments and further to paragraphs [0005]-[0011] above, the at least one therapeutic agent can include at least one hydrophobic and/or hydrophilic therapeutic agent. In accordance with these embodiments, the at least one hydrophobic and/or hydrophilic therapeutic agent, can include, but is not limited to cell proliferation agents, cell-cycle regulating agents, antisense agents, antiacids, agents against peptic ulcers and gastroesophageal reflux disease, antispasmodics, analgesics, anticholinergic drugs, propulsive drugs, antiemetics, antinausea drugs, agents for biliary therapy, agents for hepatic therapy, lipotropic agents, laxatives, anti-diarrhetic agents, intestinal adsorbents, anti-propulsive agents, anti-inflammatory drugs, agents against obesity, enzymes, hypoglycemic drugs, insulin and analogues thereof, vitamins, anabolic steroids, antithrombotic agents, antifibrinolytics, haemostatic agents, antiarrhythmic agents, cardiac stimulants, cardiac glycosides, vasodilators, antiadrenergic agents, antihypertensive drugs, diuretics, potassium-saving agents, anti-hemorrhoidals, capillary stabilizing agents, agents which act on the renin-angiotensin system, beta-blockers, selective calcium-channel blockers, non-selective calcium-channel blockers, angiotensin-converting-enzyme inhibitors, angiotensin II inhibitors, antihistamines, anesthetics, chemotherapeutic agents, anti-immune agents, corticosteroids, antiseptics, anti-acne agents, products for gynecological use, oxytocic agents, androgen, estrogen, estradiol, progestogen, progesterone, ovulation stimulants, gonadotropins, antiandrogens, drugs used in benign prostatic hypertrophy, hormones, hormone antagonists, antibiotics, antivirals, immune serum, immunoglobulins, antincoplastic agents, immunomodulatory agents, alkylation agents, antimetabolites, plant alkaloids and other natural products, cytotoxic antibiotics, immunosuppressive agents, agents for treating disorders of the musculoskeletal system, antirheumatics, muscle relaxant agents, agents which affect bone structure and mineralization, neurological agents, opioids, anti-migraine agents, anti-convulsant agents, anticholinergic agents, dopaminergic agents, antipsychotics, anxiolytics, hypnotics, sedatives, antidepressants, psychostimulants, anti-dementia agents, parasympathomimetic agents, anti-addictive disorder agents, anti-vertigo agents, antiparasitic agents, ophthalmic active ingredients, ontological active ingredients, anti-glaucoma drugs, miotics, mydriatics, cycloplegics, anti-inflammatory agents, and combinations thereof. In certain embodiments, the at least one therapeutic agent embedded in the inner core of PCL nanocapsules for delivery to a targeted cell population can include a peptide (e.g., peptide δV1-1, a specific protein kinase Cd (PKCd) inhibitor) for inducing production of or reducing loss of the targeted cell population (e.g., b-cells). In accordance with these embodiments, hydrophilic and hydrophobic therapeutic agents (cargo) are embedded in the inner core of PCL nanocapsules in different compatible compositions.

In certain embodiments and further to paragraphs [0005]-[0012] above, the at least one positively charged surfactant for binding to the at least one targeting agent can include, but is not limited to, at least one of benzalkonium chloride (BKC). In some embodiments, the at least one positively charged surfactant binds to at least one negatively charged agent which further binds to at least one targeting agent where the at least one targeting agent directs the polymer-coated nanocapsules (e.g., PCL NCs) to a targeted cell population, tissue, or other location within a subject wherein the PCL NCs are taken up by the cells, tissue, or other location of the subject. In accordance with these embodiments, the at least one therapeutic agent embedded in the PCL NCs is released after uptake.

Some embodiments and further to paragraphs [0005]-[0013] above disclosed herein include compositions that can include at least one polymer-coated nanocapsule where the at least one polymer-coated nanocapsule further includes at least one excipient. In accordance with these embodiments, the composition can be a pharmaceutical composition and further include a pharmaceutically acceptable excipient or solution for delivering to a subject.

Other embodiments disclosed herein and further to the preceding paragraphs concern kits. In accordance with these embodiments, kits disclosed herein can include at least one polymer-coated nanocapsule and at least one container. In certain embodiments, kits disclosed herein can include components for generating specific polymer-coated nanocapsules directed to bind to a target.

In yet other embodiments and further to paragraphs [0005]-[0015] above methods for targeting at least one targeted cell can include introducing at least one nanocapsule-containing composition to a subject where at least one of the at least one targeting agents includes at least one targeting agent capable of specifically binding to the at least one targeted cell or tissue and inducing a response to the at least one therapeutic agent in the subject. In accordance with these methods, the at least one targeted cell can include, but is not limited to, for example, pancreatic cells including α-cells, β-cells, δ-cells, PP-cells, and exocrine cells, brain cells, muscle cells, cardiac cells, gastrointestinal cells, liver cells, lung cells, skin cells, kidney cells, tumor cells, endometriotic cells, immune-cells, eye cells, vascular cells, ovarian cells, uterine cells, testicular cells, spleen cells or other targetable cells.

In other embodiments and further to paragraphs [0005]-[0016] above, methods for targeting at least one targeted cell or tissue in a subject to treat, reduce onset or prevent a health condition in the subject can include administering a pharmaceutical composition including at least one polymer-coated nanocapsule disclosed herein to the subject and inducing a response to the cargo encased in the polymer-coated nanocapsule in the subject to treat, reduce onset, or prevent the health condition. In some embodiments, a health condition can include at least one of an autoimmune condition, cancer, an infection, an inflammatory condition; including but not limited to, type 1 diabetes, type 2 diabetes, maturity onset diabetes of the young (MODY, all forms), insulin resistance, obesity, pancreatitis, liver injury, neural degenerative disease, heart disease or other cardiac condition, Crohn's disease, irritable bowel syndrome, ulcerative colitis or other inflammatory condition, indigestion, uterine fibroids, endometriosis or other targetable health condition or disease.

Some methods disclosed herein concern methods for creating at least one nanocapsule of use in compositions and methods disclosed. In accordance with these embodiments, a nanocapsule of use herein can include obtaining at least one liquid fatty acid and combining the at least one liquid fatty acid in solution with at least one hydrophobic (e.g., peptide) or other therapeutic agent contemplated herein). Introducing the at least one liquid fatty acid and at least one hydrophobic agent to a composition including at least one polymer and at least one cationic surfactant and agitating the combine composition. Allowing the combination composition to incubate for a period of time where organic solvent evaporates leaving behind polymer-coated nanocapsules having at least one cationic surfactant on the surface and at least one therapeutic agent in an inner fatty-acid-containing core. In certain embodiments, the at least one negatively charged agent can be introduced to the at least one cationic surfactant on the surface of the polymer-coated nanocapsules. In certain embodiments, nanocapsules can be rinsed with at least one aqueous buffer solution. In accordance with these embodiments, the at least one aqueous buffer solution can include, but is not limited to, deionized water, PBS, saline, Ringer solution or the like (e.g., one time or more) using centrifugation and removal of buffer from pelleted polymer-coated nanocapsules disclosed herein. In some embodiments, nanocapsules can be dispersed in a solution of the same or different buffer as used for a rinsing step prior to introducing at least one of a negatively charged agent and at least one targeting agent or at least one targeting agent to the polymer coated nanocapsule's cationic surfactant surface. In some embodiments, nanocapsules synthesized without the addition of a negatively charged agent or targeting agent can be further supplemented with a peptide for increased stability in a buffer solution (e.g., about 1% to about 20% w/v peptide, (e.g., bovine serum albumin, human scrum)).

In some embodiments and further to paragraphs [0005]-[0018] above, the at least one therapeutic agent can include at least one immunogenic agent. In certain embodiments, the at least one immunogenic agent can be stabilized or in a form capable of being embedded in the liquid fatty acid core of the polymer-coated NCs disclosed herein. In accordance with these embodiments, an immunogenic agent can include one or more antigens, for example a viral antigen, a bacterial antigen, a toxin, a fungal antigen, or a combination thereof. In some embodiments, the at least one immunogenic agent can also include but is not limited to, a recombinant peptide, a recombinant protein, a peptide derived from a target protein or pathogen, a synthetic peptide or protein, a virus-like particle, a live virus, a live, attenuated virus, an inactivated virus, or a combination thereof.

In certain embodiments, the at least one targeting agent disclosed at least in paragraphs [0005]-[0019] above can include at least one peptide for targeting a cell population or organ or other target in a subject. In accordance with these embodiments, the at least one peptide can further include at least one cell-penetrating peptide (CPPs) or a protein transduction domain (PTD) linked to the at least one peptide to improve transfer of the peptide through a barrier such as crossing into a cell or crossing into the brain or entering the nucleus of a cell or other transducing peptide. In certain embodiments, a PTD can include, but is not limited to, polycationic peptides (e.g., polylysine, polyarginine and polyornithine), tat, modified tatHSV-1 VP22, antp, other tat-modified molecules, or fusion peptides having two or more PTDs or CPPs or the like. In some embodiments, the at least one targeting agent can target polymer coated nanocapsules carrying cargo disclosed herein and upon reaching the targeted cell or tissue, the cargo carrying nanocapsules can be engulfed by the cells, tissues or organ and release the cargo to treat the subject having or developing a health condition (e.g., diabetes).

In some embodiments, the concentration range of the therapeutic agents in the inner core is determined by the solubility of the agent. In some embodiments, volume of the at least one liquid fatty acid can be about 1.0 ml to about 200.0 ml (or about 50.0 to about 70.0 ml), concentration of the at least one biodegradable polymer can be about 5% to about 50% w/v (or about 10% to about 40% or about 20% w/v), and concentration of the at least one cationic surfactant can be about 0.1% to about 30.0% w/v (or about 0.5% to about 20%, or about 1.0 to about 10 w/v) compared to the concentration of the at least one biodegradable polymer used and concentrations of other components that make up the polymer-coated nanocapsules. In some embodiments, polymer-coated nanocapsules disclosed herein can range from about 0.01 to about 400 nm, or about 0.1 to about 300 nm or about 0.5 to about 250 nm or less than 230 nm. In certain embodiments, polymer-coated nanocapsules disclosed herein can include sizes for effective delivery e.g., catheter or iv etc. 230 nm or less or a range that are appropriate for stabilization, storage and efficacy when delivered for example.

In some embodiments and further to paragraphs [0005]-[0021] above, each layer of the one or more coating layers can include a polymer (e.g., PCL) alone or in a suitable combination composition. In accordance with these embodiments, the outer coating layer(s) can be continuous to completely, and continuously, encapsulate the inner core containing the at least one therapeutic agent. In certain embodiments, when more than one coating layer is layered over the inner core then the more than one layer can create a completely covered nanocapsule where a second and/or third layer fills in gaps of a first coating layer, for example. In certain embodiments, one or up to several coating layers can be applied to the inner core sufficient to delay release of the at least one therapeutic agent or provide a timed-release of the at least one therapeutic agent from the inner core of the polymer-coated nanocapsule when introduced to a subject or targeted to a particular region in the subject such as a specific organ, or system for surface targeting of the one or more polymer-coated nanocapsules.

In certain embodiments, the one or more coating layer(s) disclosed herein if desired can serve as an adjuvant when an immunogenic agent or other agent with adjuvant properties can be mixed with one or more biodegradable polymer or introduced after the nanocapsules are coated to enhance an immune response in a subject against the at least one therapeutic agent(s) of the polymer-coated nanocapsules. In certain embodiments, the one or more coating layer(s) can contain a concentration capable of inducing a rapid immune response to the at least one therapeutic agent(s) of the polymer-coated nanocapsules.

In other embodiments and further to paragraphs [0005]-[0023] above, the PCL nanocapsule can further include at least a second therapeutic agent embedded in the liquid fatty acid inner core. In accordance with these embodiments, the at least the second therapeutic agent in the inner core can include at least a second therapeutic agent directed to treat, prevent, or reduce the onset of one or more health conditions (e.g., Type 1 Diabetes, cancer such as solid tumors, cardiac condition). In some embodiments, the one or more therapeutic agent or the at least second therapeutic agent embedded in the liquid fatty acid inner core of the PCL nanocapsule can be in any form including, but not limited to, a single chemical or small molecule, a stabilized therapeutic agent in a stabilizing formulation or essentially dry formulation, a time-released formulation in the form of a microparticle or slow-release formulation or a rapid-release formulation. In certain embodiments, the at least second therapeutic agent is the same or different than a first therapeutic agent. In certain embodiments, the at least second therapeutic agent is directed to treat the same or different health condition or directed to target the same or different pathway or system of the targeted cell population, organ, or system in a subject than a first therapeutic agent.

In some embodiments and further to paragraphs [0005]-[0024] above, the at least one targeting agent can be a targeting agent designed to target a tumor in a subject. In accordance with these embodiments, the at least one targeting agent can be designed to target a solid tumor in a subject. In certain embodiments, the at least one targeting agent is personalized to the subject's solid tumor to efficiently deliver the one or more therapeutic agent to the subject's solid tumor to reduce or eliminate the solid tumor in the subject.

In some embodiments and further to paragraphs [0005]-[0025] above, polymer-coated nanocapsules described herein can be stored with refrigeration for about a few weeks, about a week, for a few days or for about 1-2 days. In other embodiments, polymer-coated nanocapsules described herein can be flash frozen spray-dried, lyophilized or freeze dried and stored for a day, a few weeks, a month, or up to a year or more, or at least for one to about six months at room temperature. In certain embodiments, freeze dried polymer-coated nanocapsules can be stable after freeze-drying and rehydration in a suitable buffer with or without stabilizing agents. In certain embodiments, polymer-coated nanocapsules described herein can be stored without refrigeration up to about 50° C. to about 60° C. up to several hours without negative effect on the polymer-coated nanocapsules (e.g., without degradation or leaching of the at least one therapeutic agent from the inner core). In some embodiments, cargo-containing nanocapsules disclosed herein can be formulated for storage in a solution containing at least one sugar alcohol, surfactant, disaccharide alone or in combination with other agents. In accordance with these embodiments, cargo-containing nanocapsules disclosed herein can be formulated for storage or lyophilization and storage in a solution containing one or more of mannitol, trehalose or the like. In certain embodiments, cargo-containing nanocapsules can be frozen at −20° C. for about an hour up to about 24 hours (e.g., about 12 hours) followed by exposure to a temperature of about −80° C. (e.g., a step-down freezing method) for about 30 minutes to about 48 hours (e.g., about 24 hours), then lyophilized to an essentially dried state for storage for about an hour up to about 6 months (e.g., about a week to about one month). In accordance with these embodiments, these essentially dried cargo-containing nanocapsules can be reconstituted into a formulation suitable to maintain integrity of the cargo-containing nanocapsules for delivery to a subject.

Other embodiments, and further to paragraphs [0005]-[0026] above, provide for compositions or pharmaceutical compositions including a plurality of polymer-coated cargo-containing nanocapsules described herein. In certain embodiments, the polymer-coated cargo-containing nanocapsule-containing compositions can include polymer-coated cargo-containing nanocapsules in a pharmaceutically acceptable excipient to make a pharmaceutically acceptable composition. In accordance with these embodiments, polymer-coated cargo-containing nanocapsule-containing compositions described herein are capable of preventing, reducing the risk of onset or treating a health condition when administered to a subject.

In some embodiments and further to paragraphs [0005]-[0027] above, the polymer-coated nanocapsule-containing composition can be a single-administration polymer-coated nanocapsule-containing composition that includes one or more therapeutic agent for treating a health condition. In accordance with these embodiments, the polymer-coated nanocapsule-containing composition can be used to deliver therapeutic agents to a targeted region of a subject having one or more doses contained in the delivered polymer-coated nanocapsules. In one embodiment, the polymer-coated nanocapsule-containing nanocapsules in the composition can be similarly coated by one or more coating layers or include a mixture of polymer-coated nanocapsules of therapeutic agents where the outer coating layer of the polymer-coated nanocapsules varies in number of layers to produce a staggered release of therapeutic agents or a priming-like delivery followed by a boost delivery of at least one therapeutic agent. In certain embodiments, the priming dose of the at least one therapeutic agent can be the same or different than subsequent doses in the layered polymer-coated nanocapsules.

In some embodiments and further to paragraphs [0005]-[0028] above, the polymer-coated nanocapsule-containing composition can be a single administration composition capable of treating, preventing or reducing the risk of onset of a health condition. In certain embodiments, the single administration composition can include a single therapeutic agent or can include two, three, four or more different therapeutic agents embedded in the inner core. In other embodiments, when the two or more different therapeutic agents are present, the two or more different therapeutic agents can be contained together in a single representative polymer-coated nanocapsule having a single selected number of outer coating layers. In other embodiments, when the two or more different therapeutic agents are present, the two or more different therapeutic agents can be contained in two or more different representative polymer-coated nanocapsules having two or more different number of outer coating layers for delayed or varied timed delivery of the two or more different therapeutic agents. In certain embodiments, a single therapeutic agent can be encapsulated in polymer-coated nanocapsules disclosed herein having the same number of outer coating layers or different numbers of outer coating layers for delayed or varied timed delivery of the single therapeutic agents.

Other embodiments provide for methods for eliciting a response in a subject to the one or more therapeutic agents encapsulated in one or more polymer-coated cargo-containing nanocapsules, where the method can include administering a pharmaceutical composition described herein to the subject. In accordance with these embodiments, the pharmaceutical composition containing the one or more polymer-coated cargo-containing nanocapsules can be introduced to a subject and target a particular region or predetermined cell population or tumor of the subject and treat the subject.

Yet other embodiments provide for kits that can include at least one polymer-coated nanocapsule described herein and at least one container. In other embodiments, kits disclosed herein can include components for generating polymer-coated nanocapsules or kits for storing polymer-coated nanocapsules.

### BRIEF DESCRIPTION OF THE FIGURES

The accompanying drawings are incorporated into and form a non-limiting part of the specification to illustrate several examples of the present disclosure.

FIGS. 1A-1C represents a schematic diagram of representative polymer-coated nanocapsules having at least one targeting agent directly binding to the at least one cationic surfactant (1A); at least one targeting agent binding to the at least one negatively charged agents that binds to at least one cationic surfactant (1B) and a polymer-coated nanocapsule containing at least a second therapeutic agent (1C) according to some embodiments of the present disclosure.

FIG. 2 illustrates a schematic where a composition contains at least 3 different polymer-coated nanocapsules in a single composition according to certain embodiments disclosed herein.

FIG. 3 illustrates a schematic diagram demonstrating the components of an exemplary polymer coated nanocapsule disclosed herein and engulfment of the polymer-coated nanocapsule when the polymer-coated nanocapsule reaches its target and is endocytosed according to certain embodiments disclosed herein.

FIG. 4 is a bar graph illustrating control versus cytokine induced apoptosis in the presence and absence of an inhibitor of cytokine induced apoptosis of some embodiments of the present disclosure.

FIG. 5A is a schematic outlining water in oil (W/O) synthesis of NCs according to certain embodiments of the present disclosure where the cargo of interest is stored in an oil core surrounded by a polycaprolactone (PCL) polymer shell coated with cationic surfactant (e.g., benzalkonium chloride (BKC)). The oil phase can be combined with the aqueous phase under vigorous stirring for NC precipitation.

FIGS. 5B-5D depict representative TEM (FIG. 5B) and SEM (FIG. 5C) images of blank (no cargo) W/O NCs and a representative confocal (BODIPY encapsulated) image of W/O NCs loaded with BODIPY (FIG. 5C) according to various aspects of the present disclosure.

FIG. 6A is a schematic outlining water in oil in water (W/O/W) synthesis of NCs according to certain embodiments of the present disclosure. The primary emulsion (W1:O) consists of the cargo of interest dissolved in DI water combined with the oil phase under sonication. The primary emulsion was combined with a secondary aqueous phase under sonication (W1:O)/W2. In this example, these compositions were combined with an organic solvent containing the polymer and surfactant under rigorous stirring.

FIGS. 6B-6D depict representative SEM images of uncoated (FIG. 6B) and BSA coated (FIG. 6C) blank W/O/W NCs and a representative confocal image of W/O/W NCs loaded with Rh123 (FIG. 6D), according to various embodiments of the present disclosure.

FIGS. 7A-7C illustrate how cationic surfactant (BKC) concentration (FIG. 7A), oil core volumes (FIG. 7B), and rates of stirring (FIG. 7C) affect W/O NC size and uniformity (measured by polydispersion index (PDI). Conditions that resulted in lowest NC sizes and PDI values are noted with at least one asterisk. Values are ranked as follows; triple asterisk (**)—lowest average values, double asterisk (**)—second or third lowest values if more than four values for NC size and PDI, single asterisk (*)—second highest value for NC size and PDI, and no asterisk for highest values according to various aspects of the present disclosure.

FIG. 8 provides a table illustrating how different W/O ratios and W/O:W ratios can alter W/O/W NC size according to certain aspects of the present disclosure. Values are ranked as followed; triple asterisk (***)—lowest average value, double asterisk (**)—values within 10 nm of lowest value for NC size and 0.01 for PDI, single asterisk (*)—values within 50 nm of lowest value for NC size or 0.05 for PDI, and no asterisk-values over 50 nm of lowest value for NC size or over 0.1 for PDI.

FIGS. 9A-9C provides tables illustrating influence of different encapsulated cargos on the size of W/O NCs (FIG. 9A) and W/O/W NCs (FIG. 9B) as well as the percent of cargo that was successfully encapsulated (encapsulation efficiency) for BODIPY (hydrophobic) in W/O and rhodamine (hydrophilic) for W/O/W NCs (FIG. 9C), according to certain aspects of the present disclosure. The coefficient of determination (R2) illustrates the fit of the standard curve to the actual encapsulation efficiency data according to various aspects of the present disclosure.

FIGS. 10A-10C depicts an illustrative TEM image of BSA (5 mg/ml) coated W/O NCs with a zoomed in NC highlighting the width of the BSA coating (FIG. 10A), the Zeta potential of illustrative W/O NCs that were either uncoated or coated with BSA (5/mg/ml) or HA (0.2 mg/ml) (FIG. 10B) and fluorescence intensity of fluorescently-tagged agent (e.g., hyaluronic acid) coated onto the NCs (FIG. 10C) according to aspects of the present disclosure.

FIG. 11 is a table illustrating the change in NC size between and size distribution of uncoated and coated (BSA and HA) W/O synthesized NCs according to certain aspects of the present disclosure.

FIGS. 12A-12B are illustrative plots of Zeta potential for blank, BODIPY, and Cy5 loaded W/O NCs coated with various concentrations of HA (FIG. 12A) and BSA (FIG. 12B) according to certain aspects of the present disclosure.

FIGS. 13A-13C are illustrative dynamic light scattering (DLS) measurements of average NC diameters before and after freeze drying with various w/v mannitol (FIG. 13A) and SEM images of NCs before (FIG. 13B) and after (FIG. 13C) freeze drying and reconstituting, according to various aspects of the present disclosure.

FIGS. 14A-14C are illustrative DLS measurements of W/O NCs that were uncoated or coated with BSA (5 mg/ml) or HA (0.2 mg/ml) before and after application of mechanical shear via extrusion through a syringe (FIG. 14A), or after incubation in DMEM media for 1 or 24 hours at 37° C. (FIG. 14B) and room temperature (FIG. 14C) according to various aspects of the present disclosure.

FIGS. 15A-15B are illustrative in vitro release profiles of W/O NCs loaded with BODIPY (FIG. 15A) and W/O/W NCs loaded with Rh123 (FIG. 15B) in 1×PBS at pH 7.4, 6, and 5 according to various aspects of the present disclosure.

FIGS. 15C-15H illustrate representative SEM images of W/O NC degradation (FIG. 15C-FIG. 15E) and W/O/W NC degradation (FIG. 15F-15H) at day 0, 28 and 60, as indicated, in 1×PBS at pH 7.4, according to various aspects of the present disclosure.

FIG. 16 depicts representative confocal images of intact human islets cultured with vehicle (control) or coated Cy5-NCs (as indicated) at a concentration of 500 μl per 3.5 ml RPMI media for 24 hr, according to certain embodiments disclosed herein. Intact human islets were stained for insulin+ (green) cells, live (blue) cells, and Cy5-NCs (red).

FIG. 17 depicts representative confocal images of dissociated human islets cultured with vehicle or coated Cy5-NCs at a concentration of 125 μl per 1 ml RPMI media for 24 hr according to various aspects of the present disclosure. Images show insulin+ (green) cells, live (blue) cells, and Cy5-NCs (red).

FIG. 18 is a representative plot showing percentage of NC+ and insulin+ cells in intact and dissociated human islets cultured with Cy5-NCs with various coatings (ENTPD3 or HA-Ex4) for 24 hr according to various aspects of the present disclosure. Islets were cultured with a NC concentration of 125 μl/ml.

FIG. 19 depicts illustrative flow cytometry plots showing quantification of Cy5 NC signal and insulin positive cells via green fluorescent protein expression driven by the insulin promoter (Ins.GFP) in Mell stem cells according to some aspects of the present disclosure.

FIGS. 20A-20B depict representative confocal images showing stem cell derived beta cells (sBCs) that were untreated (FIG. 20A) or loaded with Cy5 loaded NCs for 24 hr (FIG. 20B) according to various aspects of the present disclosure.

FIGS. 20C-20D depict representative confocal images of live (NuBlue, blue) and dead (PI, red) stem cell derived beta cells (sBC) that were untreated (FIG. 20C) or treated (FIG. 20D) with PTM loaded ENTPD3 coated NCs according to various aspects of the present disclosure.

FIGS. 20E-20H depict representative graphs demonstrating percentage of dead cells for each NC treatment at 24 hr (FIG. 20E) and 72 hr (FIG. 20F) and the percentage of GFP+ dead cells over all dead cells at 24 hr (FIG. 20G) and 72 hr (FIG. 20H) according to various aspects of the present disclosure.

FIGS. 21A-21D depict representative confocal images of human islets following 24 hours of culture with vehicle (FIG. 21A) or Cy5 loaded HA-Ex4 coated NCs (FIG. 21B) or following 24 hours of culture with vehicle (FIG. 21C) or pentamidine (PTM) loaded HA-Ex4 coated NCs (FIG. 21D) according to certain aspects of the disclosure. Islets were stained for live (blue), dead (red), and insulin+ (green) cells.

FIGS. 21E-21F depict representative graphs illustrating the percentage of dead cells for each NC treatment at 24 hr (FIG. 21E) and 72 hr (FIG. 21F), according to various aspects of the present disclosure.

FIGS. 21G-21H depict representative graphs illustrating the percentage of FluoZin3+ dead cells over all dead cells at 24 hr (FIG. 21G) and 72 hr (FIG. 21H) according to various aspects of the present disclosure.

FIGS. 22A-22B depict representative confocal images (FIG. 22A) of frozen pancreatic tissue sliced from NOD-scid mice injected either with PBS only, Cy5 loaded HA-coated NCs, and Cy5 loaded HA-Ex4-loaded NCs and stained for cell nuclei (blue), insulin (green), and NCs (red), and a representative graph (FIG. 22B) quantifying the percentage of NC+ cells per total cells in fixed pancreatic islet tissue, according to certain aspects of the present disclosure.

FIGS. 22C-22D depict representative confocal images (FIG. 22C) of frozen ovary and splenic tissue sliced from NOD-scid mice injected either with PBS only, Cy5 loaded HA-coated NCs, and Cy5 loaded HA-Ex4-loaded NCs and stained for cell nuclei (blue), insulin (green), and NCs (red), and a representative graph (FIG. 22D) quantifying the percentage of NC+ cells per total cells in fixed ovarian and spleen tissue according to various aspects of the present disclosure.

### DETAILED DESCRIPTION

In the following sections, various exemplary compositions and methods are described to detail various embodiments. It will be obvious to one skilled in the art that practicing the various embodiments does not require the employment of all or even some of the specific details outlined herein, but rather that concentrations, times, and other specific details may be modified through routine experimentation. In some embodiments, well known methods or components have not been included in the description.

Nanomedicine is a fast-growing field focused on the development of nano-scale drug delivery vehicles. Targeted drug delivery using nanocapsules (NCs) across biological barriers remains a challenge limiting the effectiveness of these treatments. Embodiments of the present disclosure provide novel compositions, methods of use and methods for making targeted therapeutic agent-containing coated nanocapsules. In certain embodiments, the present disclosure provides compositions and methods for generating and using the targeted therapeutic agent-containing coated nanocapsules to treat, reduce onset, or prevent a health condition in a subject. In some embodiments, a nanocapsule includes but is not limited to, an inner core of at least one liquid fatty acid core containing at least one therapeutic agent, a biodegradable polymer coating the inner core, at least one positively charged surfactant and at least one targeting agent associated with the at least one positively charged surfactant.

Polycaprolactone (PCL), a biodegradable and biocompatible polymer, has been used for drug delivery strategies. PCL nanospheres have previously been functionalized with hyaluronic acid through incorporation of a cationic surfactant into the polymer shell. This strategy has shown improved nanosphere circulation time; however, this technique has not been performed using nanocapsules and further have not been designed to specifically target a cell population or other region in a subject as disclosed herein

Embodiments of the instant disclosure creates and tests a selected system for specific targeting and delivery of pre-selected therapeutic agents as a proof of concept of the instantly claimed targeting polymer coated nanocapsules to cells and tissues in subject for targeted treatment or prevention of a health condition. In order to verify the success of these targeting polymer-coated nanocapsules disclosed herein, a novel nanocapsule design that includes a liquid fatty acid inner core containing at least one therapeutic agent and a polymer shell that incorporates positively charged surfactant on the surface that can adsorb targeting peptides or other molecules to either directly or via conjugation through a negatively charged agent (e.g., hyaluronic acid), permitting specific targeting and endocytosis of the polymer-coated nanocapsules and their cargo to β-cells or any other targeted site in a subject. This proof of concept is supportive of a plug and play model for directly targeting any cell or other region in a subject with delivery of any therapeutic agent capable that can be made compatible and stable in a liquid fatty acid composition core for treating or preventing a health condition.

Embodiments of the present disclosure provide novel compositions and methods for making and using polymer-coated nanocapsules. In certain embodiments, compositions and methods are disclosed for embedding at least one therapeutic agent in a composition including at least one fatty acid to form an inner core; at least one layer of a shell coating the inner core, the coating layer including at least one biodegradable polymer to make a biodegradable polymer shell coating. In accordance with these embodiments, the at least one coating layer of the at least one biodegradable polymer shell coating further includes at least one positively charged surfactant on the surface or in pores of the at least one biodegradable polymer shell coating layer(s); and at least one targeting agent associated with at least one of the at least one positively charged surfactant. Alternatively, at least one negatively charged agent can be associated with the at least one positively charged surfactant and the at least one negatively charged agent can be associated with or bind to the at least one targeting agent for linking the at least one targeting agent to the polymer-coated nanocapsules.

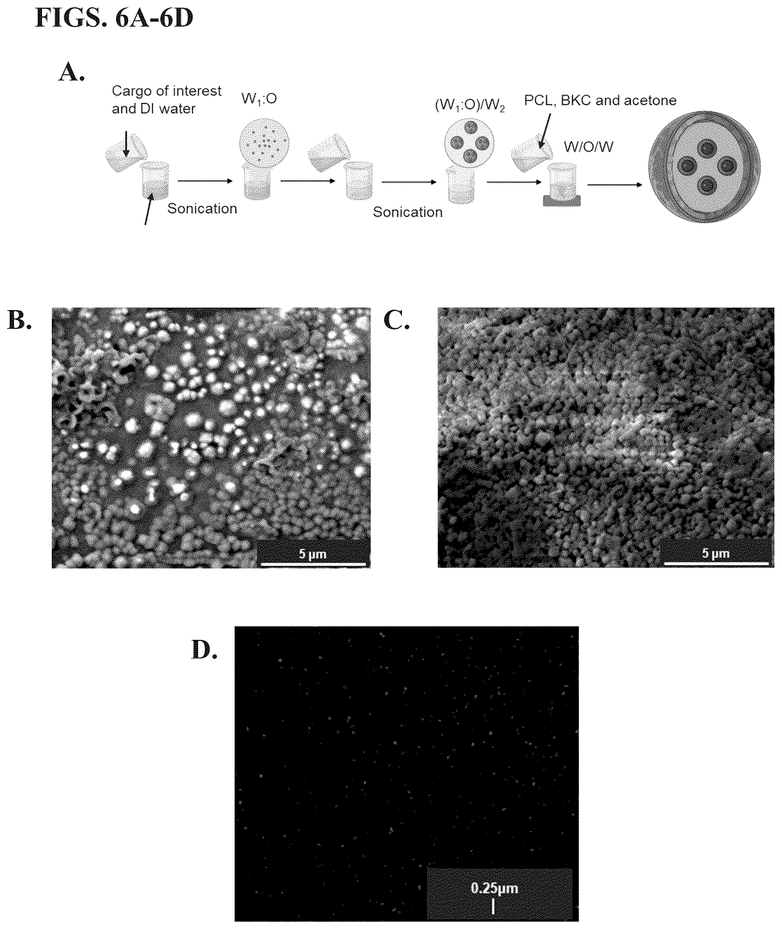

In certain embodiments and further to paragraphs [0064]-[0067] above, the at least one therapeutic agent comprises at least one hydrophilic therapeutic agent. In certain embodiments, a hydrophilic therapeutic agent can be contained within nanocapsules using a double emulsion procedure as disclosed herein. In accordance with these embodiments, the at least one hydrophilic therapeutic agent can be dissolved in a solution prior to being combined with the at least one fatty acid; for example, to create an internal aqueous environment. Solutions compatible with the at least one hydrophilic therapeutic agent can include water, PBS, saline, dextrose, Ringer's solution, or Lactated Ringer's solution or other suitable solution for creating a hydrophilic therapeutic agent-containing composition for introducing to the one or more liquid fatty acid. In certain embodiments, the combination of the solution and the at least one hydrophilic therapeutic agent can remain as a droplet within the fatty acid inner core where the at least one fatty acid provides a coating around the combination of the solution and the at least one hydrophilic therapeutic agent (See for example FIG. 6A, for example).

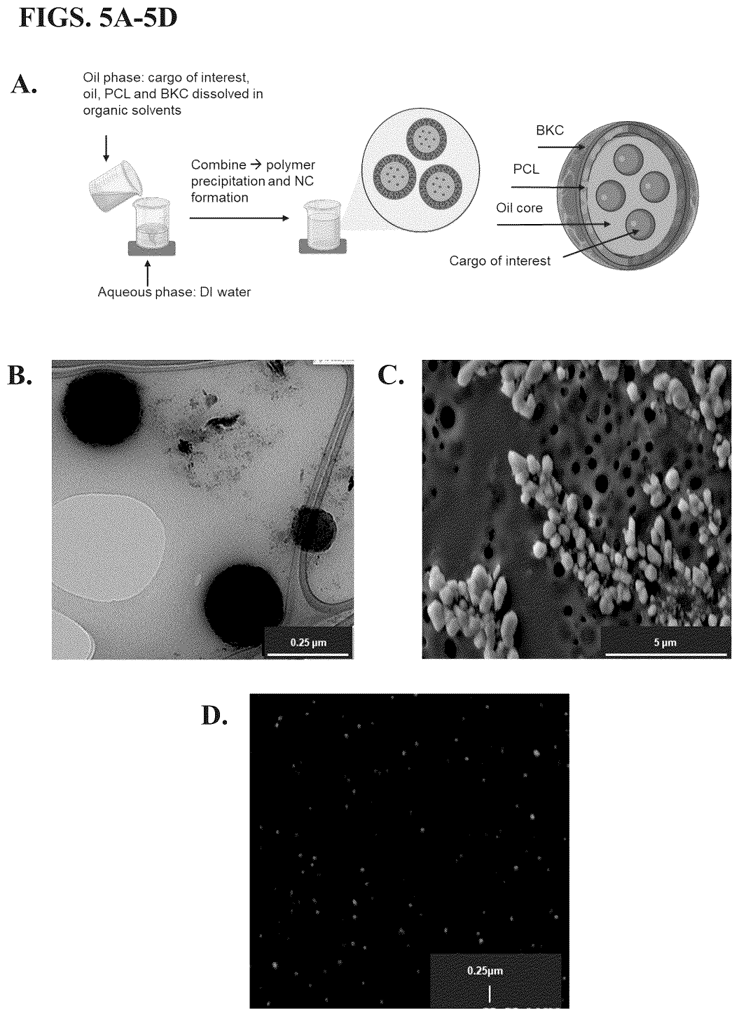

In certain embodiments and further to paragraphs [0064]-[0068] above, the at least one therapeutic agent comprises at least one hydrophobic therapeutic agent. In certain embodiments, the at least one hydrophobic therapeutic agent can be contained within nanocapsules using a single emulsion procedure as disclosed herein. In accordance with these embodiments, the at least one hydrophobic therapeutic agent can be combined directly with the at least one fatty acid for dispersion within the at least one fatty acid inner core (See FIG. 5A, for example). In some embodiments, an inner core of a polymer coated nanocapsule disclosed herein can include mixtures of at least one hydrophilic agent, at least one hydrophobic agent or mixtures thereof. In other embodiments, nanocapsules containing at least one hydrophilic agent and a hydrophobic agent can be generated by methods disclosed herein using single and double emulsion procedures disclosed herein (e.g., by dissolving the hydrophilic agent in an aqueous solution before combining with a fatty acid dispersion containing the hydrophobic agent). In other embodiments, hydrophobic therapeutic agent-containing nanocapsules can be mixed with hydrophilic therapeutic agent-containing nanocapsules in a single composition for administration to a subject. In other embodiments, a particular therapeutic agent for delivery to a targeted region disclosed herein can be encased in or mixed with another stabilizing agent such as a surfactant such as high or low molecular weight surfactant, emulsifiers, gelatins, detoxifying agents, anti-microbial agents, adjuvants, inactivating agents, alcohols, poloxamers, tweens, other polymers, salts, pH balancing solutions, water, a saline solution, or other agents for stabilizing and prolonging the integrity and reducing degradation of the therapeutic agent. Advantages of polymer-coated nanocapsules disclosed herein include, but are not limited to, permitting high loading capacity, mixed loading capacity, improved efficacy of treatment, reduced loss of therapeutic agent due to targeted delivery, reduction of leakage of the at least one therapeutic agent during storage, transport and administration of the one or more therapeutic agent contained in polymer-coated nanocapsules.

In some embodiments and further to paragraphs [0064]-[0069] above, the nanocapsules can be coated with at least one biodegradable, biocompatible polymer can include, but is not limited to, at least one of polycaprolactone (PCL), poly(lactic-co-glycolic acid) (PLGA), polylactic acid (PLA), chitosan, gelatin, and a biodegradable polymer having a monomer size ranging from about 0.1 kDa to about 80 kDa. In some embodiments, the at least one biodegradable polymer includes at least one polymer of polycaprolactone (PCL). In other embodiments, the at least one biodegradable, biocompatible polymer can further include the at least one positively charged surfactant on the surface or in a pore of the at least one biodegradable, biocompatible polymer shell coating layer. In certain embodiments, the at least one biodegradable, biocompatible polymer can further include the at least one positively charged surfactant on the surface or in a pore of the at least one biodegradable, biocompatible polymer shell coating layer further includes at least one negatively charged agent, where the at least one negatively charged agent is capable of associating with or linking to the at least one positively charged surfactant and the at least one targeting agent. In accordance with these embodiments, the at least one negatively charged agent can include at least one of hyaluronic acid, aggrecan, versican, syndecan, nidogen, decorin, biglycan, chondroitin sulfate, keratin sulfate, γ-polyglutamic acid, oligocthylene glycol, or other negatively charged protein or brush polymer known in the art.

In other embodiments and further to paragraphs [0064]-[0070] above, the polymer coated nanocapsule according to any of the preceding paragraphs can include an inner core of at least one liquid fatty acid composition (e.g., oil or liquid lipid) for example, made up of at least one liquid lipid composition. In certain embodiments, the at least one liquid fatty acid is made up of at least one naturally-occurring biocompatible liquid fatty acid. In some embodiments, the at least one naturally-occurring biocompatible liquid fatty acid can include a fatty acid that is liquid at room temperature (e.g., 20-25° C. and/or a melting point below 4° C.) or can be a semi-liquid. In certain embodiments, the at least one liquid fatty acid that makes up at least part of the inner core disclosed herein includes at least one of: coconut oil, sunflower oil, vegetable oil, soybean oil, colza oil, peanut oil, mineral oil, corn oil, olive oil, palm oil, cottonseed oil, castor oil, linseed oil, borage oil, evening primrose oil, marine oils, fish oils, algae oils, oils derived from petroleum, liquid paraffin, short-chain fatty alcohols, medium-chain aliphatic branched fatty alcohols, fatty acid esters with short-chain alcohols, isopropyl myristate, isopropyl palmitate, medium-chain triglycerides, capric and caprylic triglycerides, and mixtures thereof. In some embodiments, the at least one fatty acid includes at least coconut oil or the like.

In certain embodiments and further to paragraphs [0064]-[0071] above, the at least one targeting agent can include at least one of a polypeptide, a polynucleotide, a chimeric molecule, a glycoprotein, a whole organism, a whole cell, a pathogen, a toxin, a polysaccharide, a small molecule, a fragment or segment thereof, a metabolite, a chemical classified as a chemical or biological agent or other pharmaceutical agent thereof.

In some embodiments and further to paragraphs [0064]-[0072] above, the at least one targeting agent can include at least one of an antibody, a ligand, a receptor, an enzyme, a viral antigen, a bacterial antigen, a yeast antigen, a prion antigen, a toxin, a recombinant peptide, a recombinant protein, a polypeptide derived from a target protein or pathogen, a synthetic peptide or protein, a polynucleotide derived from a target protein or pathogen, a virus-like particle, a live virus, a live, attenuated virus, an inactivated virus. In certain embodiments and further to paragraphs [0064]-[0072] above, the at least one targeting agent includes at least one of a polypeptide or an antibody. In certain embodiments, the at least one targeting agent that includes at least one of a polypeptide or an antibody includes a polypeptide or an antibody capable of associating with a specific molecule associated with a target cell population (e.g., pancreatic b cells, kidney cells, cardiac cells, liver cells, lung cells), a targeted organ, or a targeted region (e.g., lung, liver, gastrointestinal tract, in a subject (e.g., targeted molecule like a glucagon-like peptide-1 receptor (GLP-1R) agonist Exendin-4; antibody against a specific b-cell marker ENTPD3 (NTPDase3) or similar). In certain embodiments, a targeted cell population can be the specific target for the polymer-coated nanocapsules disclosed herein (e.g., pancreatic b cells). In accordance with these embodiments, the at least one targeting agent directs the at least one polymer-coated nanocapsules containing at least one therapeutic agent to the target in order for the at least one polymer-coated nanocapsules containing at least one therapeutic agent to be engulfed by the target, the polymer coating to degrade or dissolve and the at least one therapeutic agent contained therein treat, prevent onset or reduce risk of a health condition associated with the target (e.g., type-1 diabetes and reduction or prevention of pancreatic b cell death and/or preservation of insulin-producing b cells).

In other embodiments and further to paragraphs [0064]-[0073] above, the at least one therapeutic agent can include at least one hydrophobic and/or hydrophilic therapeutic agent or agent capable of being adapted to a formulation containing at least one fatty acid to form the inner core of the polymer-coated nanocapsules disclosed herein. In accordance with these embodiments, the at least one hydrophobic and/or hydrophilic therapeutic agent, can include, but is not limited to cell proliferation agents, cell cycle regulating agents, antisense agents and antiacids, agents against peptic ulcers and gastroesophageal reflux disease, antispasmodics, analgesics, anticholinergic drugs, propulsive drugs, antiemetics, antinausea drugs, agents for biliary therapy, agents for hepatic therapy, lipotropic agents, laxatives, anti-diarrhetic agents, intestinal adsorbents, anti-propulsive agents, anti-inflammatory drugs, agents against obesity, enzymes, hypoglycemic drugs, insulin and analogues thereof, vitamins, anabolic steroids, antithrombotic agents, antifibrinolytics, haemostatic agents, antiarrhythmic agents, cardiac stimulants, cardiac glycosides, vasodilators, antiadrenergic agents, antihypertensive drugs, diuretics, potassium-saving agents, anti-hemorrhoidals, capillary stabilizing agents, agents which act on the renin-angiotensin system, beta-blockers, selective calcium-channel blockers, non-selective calcium-channel blockers, angiotensin-converting-enzyme inhibitors, angiotensin II inhibitors, antihistamines, anesthetics, chemotherapeutic agents, anti-immune agents, corticosteroids, antiseptics, anti-acne agents, products for gynecological use, oxytocic agents, androgen, estrogen, estradiol, progestogen, progesterone, ovulation stimulants, gonadotropins, antiandrogens, drugs used in benign prostatic hypertrophy, hormones, hormone antagonists, antibiotics, antivirals, immune serum, immunoglobulins, antineoplastic agents, immunomodulatory agents, alkylation agents, antimetabolites, plant alkaloids and other natural products, cytotoxic antibiotics, immunosuppressive agents, agents for treating disorders of the musculoskeletal system, antirheumatics, muscle relaxant agents, agents which affect bone structure and mineralization, neurological agents, opioids, anti-migraine agents, anti-convulsant agents, anticholinergic agents, dopaminergic agents, antipsychotics, anxiolytics, hypnotics, sedatives, antidepressants, psychostimulants, anti-dementia agents, parasympathomimetic agents, anti-addictive disorder agents, anti-vertigo agents, antiparasitic agents, ophthalmic active ingredients, ontological active ingredients, anti-glaucoma drugs, miotics, mydriatics, cycloplegics, anti-inflammatory agents, and combinations thereof. In certain embodiments, the at least one therapeutic agent embedded in the inner core of PCL nanocapsules for delivery to a targeted cell population can include a peptide (e.g., peptide δV1-1, a specific protein kinase Cd (PKCd) inhibitor) for inducing production of or reducing loss of the targeted cell population (e.g., b-cells). In other embodiments, the at least one therapeutic agent can include a vaccine such as an anti-viral, anti-bacterial, anti-fungal, anti-protozoan or vaccine to reduce drug dependency or the like.

In certain embodiments and further to paragraphs [0064]-[0074] above, the at least one positively charged surfactant for binding to the at least one targeting agent can include, but is not limited to, at least one of benzalkonium chloride (BKC), quaternary ammonium salts, such as cetyl trimethyl ammonium bromide, lauryl trimethyl ammonium chloride, benzyl dimethyl hexadecyl ammonium chloride, distearyl dimethyl ammonium chloride, dilauryl dimethyl ammonium chloride, dimyristyl dimethyl ammonium chloride, cetylpyridinium chloride, benzethonium chloride, methyl benzetonium chloride, or the like, or mixtures or combinations thereof. In some embodiments, the at least one positively charged surfactant that optionally binds to at least one negatively charged agent associates with the at least one targeting agent where the at least one targeting agent can direct the polymer-coated nanocapsules (e.g., PCL NCs) to a targeted cell population, tissue, organ, or other location within a subject to treat, prevent or reduce onset of a condition.

Some embodiments and further to paragraphs [0064]-[0075] above, disclosed herein include compositions that can include at least one polymer-coated cargo-containing nanocapsule where the at least one polymer-coated nanocapsule further includes at least one excipient. In accordance with these embodiments, the composition can be a pharmaceutical composition and further include a pharmaceutically acceptable excipient or solution for delivering to a subject including, but not limited to, PBS, saline, dextrose, Ringer's solution or other suitable excipient.

Other embodiments disclosed herein and further to paragraphs [0064]-[0076] above, concern kits. In accordance with these embodiments, kits disclosed herein can include at least one polymer-coated nanocapsule and at least one container. In certain embodiments, kits disclosed herein can include components for generating specific polymer-coated nanocapsules directed to bind to a target. In yet other embodiments, kits disclosed herein can include instructions for making and/or using the at least one polymer-coated nanocapsule targeted to bind to a specific region, cell, tissue, or organ of a subject. In certain embodiments, kits can include at least one polymer coated nanocapsule in a composition directed to bind a particular cell type in a subject (e.g., b-cells) and at least one container. In accordance with these embodiments, kits can further include a device for administering the at least one polymer coated nanocapsule composition to a subject and instructions for performing the same.

In yet other embodiments and further to paragraphs [0064]-[0077] above methods for targeting at least one targeted cell can include introducing at least one polymer-coated nanocapsule-containing composition to a subject where at least one of the at least one targeting agents includes at least one targeting agent capable of specifically binding to the at least one targeted cell and inducing a response to the at least one therapeutic agent in the subject. In accordance with these methods, the at least one targeted cell can include, but is not limited to, for example, pancreatic cells including α-cells, β-cells, δ-cells; PP-cells, and exocrine cells, brain cells, muscle cells, cardiac cells, gastrointestinal cells, liver cells, lung cells, skin cells, kidney cells, tumor cells, endometriotic cells, immune-cells, eye cells, vascular cells, ovarian cells, uterine cells, testicular cells, spleen cells or other targetable cells.

In other embodiments and further to paragraphs [0064]-[0077] above, methods for targeting at least one targeted cell in a subject to treat, reduce onset or prevent a health condition in the subject can include administering a pharmaceutical composition including at least one polymer-coated nanocapsule (e.g., PCL NC) to the subject and inducing a response in the subject to treat, reduce onset, or prevent the health condition. In some embodiments, a health condition can include at least one of type 1 diabetes, type 2 diabetes, cancer such as a solid tumor or cancer isolated to a particular tissue, organ, or cell-type (e.g., lung, liver, kidney, brain, breast, bladder, uterine, ovarian, prostate, stomach, skin, lymph node, bone, blood), an inflammatory condition in the lungs or GI tract or isolated region, skin-related issues, hair health and growth, or combination thereof, or the like. In certain embodiments, the pharmaceutical composition including at least one polymer-coated nanocapsule can include one therapeutic agent, two therapeutic agents, three therapeutic agents, or more depending on the subject and the condition to be prevented and/or treated.

In other embodiments and further to paragraphs [0064]-[0079] above, a subject can be treated multiple times per day, twice per day, daily, every other day, twice weekly, weekly, every other week, monthly, or any regimen in between, or other regimen depending on the subject and the condition to be treated. In certain embodiments, a single administration can be used to treat a subject and a health professional determine the timing, if any, for a supplemental administration of a composition including at least, therapeutic agent-containing polymer-coated nanocapsules disclosed herein.

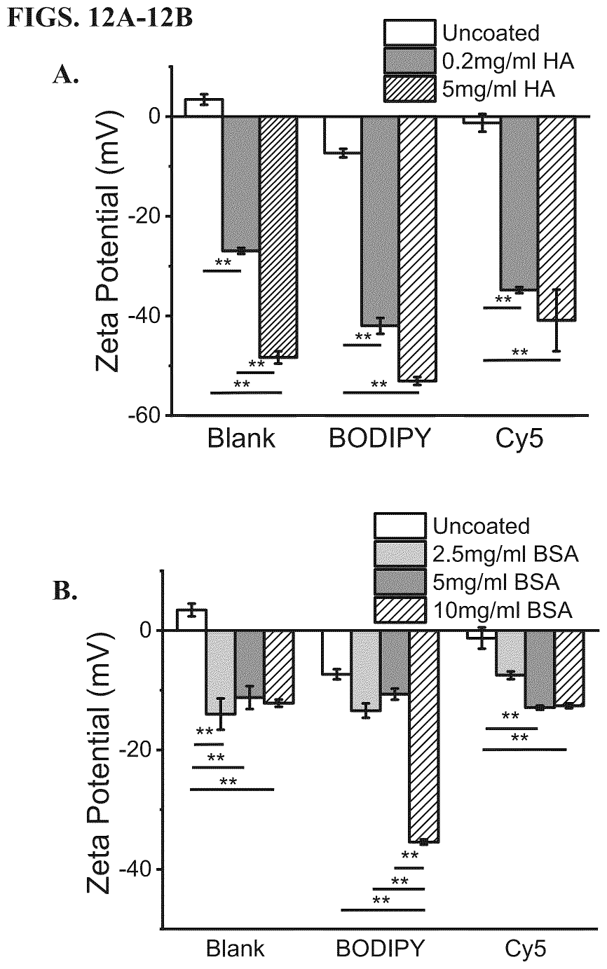

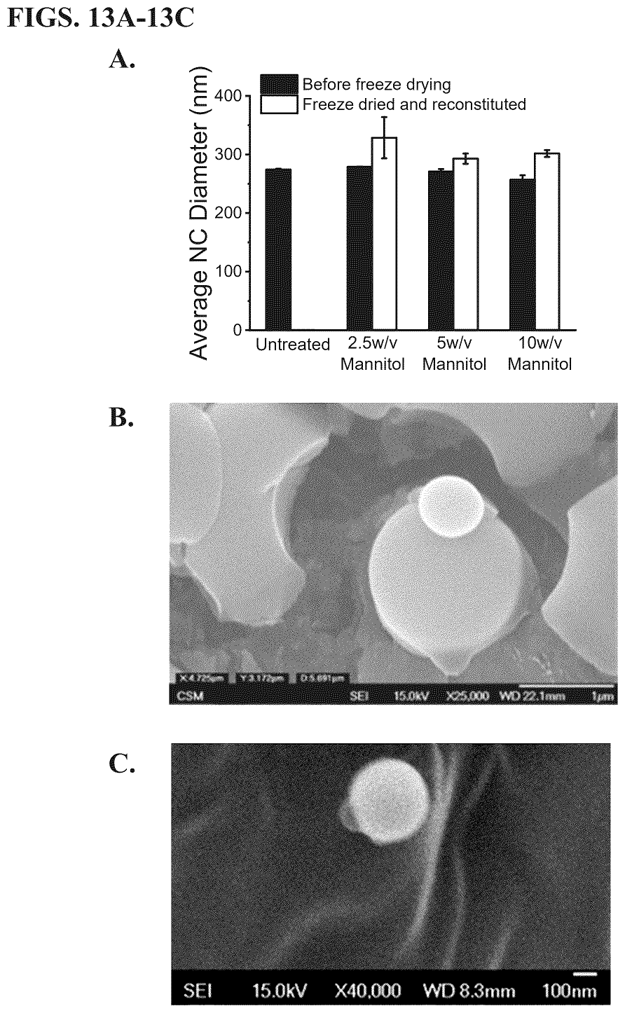

Certain embodiments disclosed herein concern methods for creating at least one polymer-coated nanocapsule of use in compositions and methods disclosed. In accordance with these embodiments, creating a polymer-coated nanocapsule of use herein can include obtaining at least one liquid fatty acid and combining the at least one liquid fatty acid in solution with at least one therapeutic agent and optionally another agent for compatibility with the one or more liquid fatty acid (e.g., hydrophobic and/or hydrophilic agent (e.g., peptide or other therapeutic agent contemplated herein) and optionally water, PBS, stabilizing composition or the like). In accordance with these embodiments, polymer-coated nanocapsule fabrication can be performed by a single emulsion for hydrophobic therapeutic agents (See for example, FIG. 12) or double emulsion for hydrophilic therapeutic agents (See for example FIG. 13) or a combined single and double emulsion for creating a mixture of hydrophilic and hydrophobic therapeutic agent containing polymer-coated nanocapsules. Introducing the at least one liquid fatty acid and at least one therapeutic agent to a composition including at least one polymer and at least one cationic surfactant and agitating the combined composition. Allowing the combination composition to incubate for a period of time under vacuum where organic solvent evaporates leaving behind polymer-coated nanocapsules having at least one cationic surfactant on the surface and at least one therapeutic agent in an inner fatty-acid-containing core. Introducing at least one of a negatively charged agent and at least one targeting agent or at least one targeting agent without a negatively charged agent to the polymer-coated nanocapsule's cationic surfactant surface. In some embodiments, polymer-coated nanocapsules disclosed herein can be collected by filtration, centrifugation, spray-drying, freeze spray drying and/or lyophilization. In certain embodiments, polymer-coated nanocapsules disclosed herein can be collected by centrifugation and/or lyophilization. In certain embodiments, polymer-coated nanocapsules can be rinsed with at least one aqueous buffer solution. In accordance with these embodiments, the at least one aqueous buffer solution can include, but is not limited to, deionized water, PBS, saline, Ringer solution, a buffer having a physiological pH or the like (e.g., at least one time, up to five times or more to thoroughly rinse the nanocapsules) using centrifugation and removal of buffer from pelleted polymer-coated nanocapsules disclosed herein. In some embodiments, nanocapsules can be dispersed in a solution of the same or different buffer as used for a rinsing step prior to introducing at least one of a negatively charged agent and at least one targeting agent or at least one targeting agent to the polymer coated nanocapsule's cationic surfactant surface. In some embodiments, polymer-coated nanocapsules synthesized without the addition of a negatively charged agent or targeting agent can be further supplemented with a peptide for increased stability in a buffer solution (e.g., about 1% to about 20% w/v peptide, or about 2.0% to about 15% w/v or about 2.5% (w/v) peptide (e.g., bovine serum albumin, human serum or other appropriate protein or peptide)).

In some embodiments and further to paragraphs [0064]-[0081] above, the at least one therapeutic agent can include at least one immunogenic agent. In certain embodiments, the at least one immunogenic agent can be stabilized or in a form capable of being embedded in the liquid fatty acid core of the polymer-coated NCs disclosed herein. In accordance with these embodiments, an immunogenic agent can include one or more antigens, for example a viral antigen, a bacterial antigen, a toxin, or a combination thereof. In some embodiments, the at least one immunogenic agent can also include but is not limited to, a recombinant peptide, a recombinant protein, a peptide derived from a target protein or pathogen, a synthetic peptide or protein, a virus-like particle, a live virus, a live, attenuated virus, an inactivated virus, or a combination thereof.

In some embodiments disclosed herein and further to paragraphs [0064]-[0082] above, the polymer-coated nanocapsules is a composition for delivery to a subject such as a human or other mammal or animal or bird (e.g., pet, livestock, horse, zoo animal or other animal or bird, reptile etc.) can include at least one additional agent in addition to an acceptable excipient. In accordance with these embodiments, the at least one additional agent can include, but is not limited to, but is not limited to cell proliferation agents, cell-cycle regulating agents, antisense agents, antiacids, agents against peptic ulcers and gastroesophageal reflux disease, antispasmodics, analgesics, anticholinergic drugs, propulsive drugs, antiemetics, antinausea drugs, agents for biliary therapy, agents for hepatic therapy, lipotropic agents, laxatives, anti-diarrhetic agents, intestinal adsorbents, anti-propulsive agents, anti-inflammatory drugs, agents against obesity, enzymes, hypoglycemic drugs, insulin and analogues thereof, vitamins, anabolic steroids, antithrombotic agents, antifibrinolytics, haemostatic agents, antiarrhythmic agents, cardiac stimulants, cardiac glycosides, vasodilators, antiadrenergic agents, antihypertensive drugs, diuretics, potassium-saving agents, anti-hemorrhoidals, capillary stabilizing agents, agents which act on the renin-angiotensin system, beta-blockers, selective calcium-channel blockers, non-selective calcium-channel blockers, angiotensin-converting-enzyme inhibitors, angiotensin II inhibitors, antihistamines, anesthetics, chemotherapeutic agents, anti-immune agents, corticosteroids, antiseptics, anti-acne agents, products for gynecological use, oxytocic agents, androgen, estrogen, estradiol, progestogen, progesterone, ovulation stimulants, gonadotropins, antiandrogens, drugs used in benign prostatic hypertrophy, hormones, hormone antagonists, antibiotics, antivirals, immune serum, immunoglobulins, antineoplastic agents, immunomodulatory agents, alkylation agents, antimetabolites, plant alkaloids and other natural products, cytotoxic antibiotics, immunosuppressive agents, agents for treating disorders of the musculoskeletal system, antirheumatics, muscle relaxant agents, agents which affect bone structure and mineralization, neurological agents, opioids, anti-migraine agents, anti-convulsant agents, anticholinergic agents, dopaminergic agents, antipsychotics, anxiolytics, hypnotics, sedatives, antidepressants, psychostimulants, anti-dementia agents, parasympathomimetic agents, anti-addictive disorder agents, anti-vertigo agents, antiparasitic agents, ophthalmic active ingredients, ontological active ingredients, anti-glaucoma drugs, miotics, mydriatics, cycloplegics, anti-inflammatory agents, other suitable agents, and combinations thereof. In certain embodiments, the at least one additional agent combined with polymer-coated nanocapsules for delivery to a targeted cell population can include a peptide (e.g., peptide δV1-1, a specific protein kinase Cd (PKCd) inhibitor) for inducing production or expansion of or reducing loss of the targeted cell population (e.g., β-cells) or combination thereof.

In certain embodiments, the at least one targeting agent disclosed at least in paragraphs [0064]-[0083] above, can include at least one peptide for targeting a cell population or organ or other target. In accordance with these embodiments, the at least one peptide can further include at least one cell-penetrating peptide (CPP) or a protein transduction domain (PTD) linked to the at least one peptide to improve transfer of the peptide through a barrier such as crossing into a cell or crossing into the brain or entering the nucleus of a cell or other transducing peptide for facilitating targeting of the polymer-coated nanocapsules disclosed herein. In certain embodiments, a PTD can include, but is not limited to, polycationic peptides (e.g., polylysine, polyarginine and polyornithine), tat, modified tatHSV-1 VP22, antp, other tat-modified molecules, or fusion peptides having two or more PTDs or CPPs or the like.

In certain embodiments and further to paragraphs [0064]-[0084] above, the concentration of hydrophilic and/or hydrophobic therapeutic agent contained in the polymer-coated nanocapsules disclosed herein can be from about 0.00001% w/v to about 50% w/v, or about 0.0001% w/v to about 40% w/v or about 0.001% in w/v to about 30% w/v or about 0.01 to about 25% w/v or other appropriate concentration depending on the subject to be treated and the targeted health condition. In some embodiments, the concentration range of the therapeutic agents in the inner core is determined by the solubility of the agent. In some embodiments, volume of the at least one liquid fatty acid can be about 1.0 ml to about 200.0 ml (or about 10.0 ml to about 150.0 ml or about 50.0 to about 70.0 ml), concentration of the at least one biodegradable polymer can be about 5.0% to about 50.0% w/v (or about 10% to about 40% or about 10 to about 25% w/v, or about 20%), and concentration of the at least one cationic surfactant can be about 0.1% to about 30.0% w/v (or about 0.5% to about 20%, or about 1.0% to about 10.0% w/v) compared to the concentration of the at least one biodegradable polymer used and concentrations of other components that make up the polymer-coated nanocapsules. In some embodiments, polymer-coated nanocapsules disclosed herein can range in size from about 0.01 nm to about 400.0 nm, or about 0.1 nm to about 300.0 nm or about 0.5 nm to about 250.0 nm or less than 230 nm. In certain embodiments, polymer-coated nanocapsules disclosed herein can include sizes for effective delivery e.g., catheter or iv etc. 250 nm or less or a range appropriate for stabilization, storage and efficacy when delivered to the subject for example.

In some embodiments and further to paragraphs [0064]-[0085] above, each layer of the one or more coating layers of biodegradable polymer can include a polymer (e.g., PCL) alone or in a suitable combination composition of polymers to create an outer coating of the inner core of the nanocapsules disclosed herein. In accordance with these embodiments, the outer coating layer(s) can be continuous to completely, and continuously, encapsulate the inner core containing the at least one therapeutic agent. In certain embodiments, when more than one coating layer is layered over the inner core then the more than one layer can create a completely covered nanocapsule where a second and/or third layer fills in gaps of a first coating layer, for example. In certain embodiments, one or up to several coating layers can be applied to the inner core sufficient to delay release of the at least one therapeutic agent or provide a timed-release of the at least one therapeutic agent from the inner core of the polymer-coated nanocapsule when introduced to a subject or targeted to a particular region in the subject such as a specific organ or system. In accordance with these embodiments, the contents in the inner core of the polymer-coated nanocapsules disclosed herein can be sufficiently coated with biodegradable polymer to reach the intended target, get encapsulated and then release the contents, including, but not limited to, releasing the at least one therapeutic agent contained therein.

In certain embodiments and further to paragraphs [0064]-[0086] above, the one or more coating layer(s) disclosed herein if desired can serve as an adjuvant when an immunogenic agent or other immunostimulating agent having adjuvant properties is mixed with one or more biodegradable polymer(s) or when an immunogenic agent or other immunostimulating agent (e.g., alum, viral antigen or other agent known in the art compatible with the selected biodegradable polymer(s) such as PCL) having adjuvant properties is introduced after the nanocapsules are coated with the at least one biodegradable polymer to enhance an immune response in a subject against the at least one therapeutic agent(s) of the polymer-coated nanocapsules. In certain embodiments, the one or more coating layer(s) of the at least one biodegradable polymer combined with an immunostimulating agent can contain a concentration capable of inducing a rapid immune response to the at least one therapeutic agent(s) of the polymer-coated nanocapsules when targeted to a preselected region of a subject.

In other embodiments and further to paragraphs [0064]-[0087] above, the PCL nanocapsule can further include at least a second therapeutic agent embedded in the liquid fatty acid inner core. In accordance with these embodiments, the at least the second therapeutic agent in the inner core can include at least a second therapeutic agent directed to treat, prevent, or reduce the onset of one or more health conditions (e.g., Type 1 Diabetes, cancer such as solid tumors, cardiac condition). In some embodiments, the one or more therapeutic agent or the at least second therapeutic agent embedded in the liquid fatty acid inner core of the PCL nanocapsule can be in any form including, but not limited to, a single chemical or small molecule, a stabilized therapeutic agent in a stabilizing formulation or essentially dry formulation, a time-released formulation in the form of a microparticle or slow-release formulation or a rapid-release formulation. In certain embodiments, the at least second therapeutic agent is the same or different than a first therapeutic agent. In other embodiments, the at least second therapeutic agent is directed to treat the same or different health condition or directed to target the same or different pathway or system of the targeted cell population, organ, or system in a subject than a first therapeutic agent.

In some embodiments and further to paragraphs [0064]-[0088] above, the at least one targeting agent can be a targeting agent designed to target a tumor in a subject. In accordance with these embodiments, the at least one targeting agent can be designed to target a solid tumor in a subject. In certain embodiments, the at least one targeting agent is personalized to the subject's solid tumor in to efficiently deliver the one or more therapeutic agent to the subject's solid tumor to more quickly shrink, reduce, impede the expansion of, or eliminate the tumor in the subject. In accordance with these embodiments, the at least one targeting agent of a personalized treatment regimen of polymer-coated nanocapsules disclosed herein can include at least one receptor targeting agent where the receptor is specific to the targeted solid tumor or region of the subject (e.g., a breast cancer, ovarian or other tumor having specific accessible markers or receptors on the surface).

In some embodiments and further to paragraphs [0064]-[0089] above, polymer-coated nanocapsules described herein can be stored with refrigeration for about a few weeks, for about a week, for a few days or for about 1-2 days. In other embodiments, polymer-coated nanocapsules described herein can be flash frozen spray-dried, lyophilized or freeze dried and stored for a day to a year or more, or at least for one to about six months, or at least for several months without refrigeration or cold storage, for example, at room temperature. In certain embodiments, freeze dried polymer-coated nanocapsules can be stable after freeze-drying and rehydration in a suitable buffer with or without stabilizing agents for several months without refrigeration or cold storage, for example, at room temperature. In certain embodiments, polymer-coated cargo-containing nanocapsules described herein can be stored without refrigeration up to about 50° C. to about 60° C. up to several hours without negative effect on the polymer-coated cargo-containing nanocapsules (e.g., without degradation or leaching of the at least one therapeutic agent from the inner core).

Other embodiments, and further to paragraphs [0064]-[0090] above, provide for compositions or pharmaceutical compositions including a plurality of polymer-coated nanocapsules described herein. In certain embodiments, the polymer-coated nanocapsule-containing compositions can include polymer-coated nanocapsules in a pharmaceutically acceptable excipient to make a pharmaceutically acceptable composition where the polymer-coated nanocapsules harbor or encapsulate one, two, three or more therapeutic agents. In accordance with these embodiments, the polymer-coated nanocapsule-containing compositions described herein are capable of preventing, reducing the risk of onset, or treating a health condition when administered to a subject.

In some embodiments and further to paragraphs [0064]-[0091] above, the polymer-coated nanocapsule-containing composition can be a single-administration or single-dose polymer-coated nanocapsule-containing composition that includes one or more therapeutic agent for treating a health condition. In accordance with these embodiments, the therapeutic agent-containing polymer-coated nanocapsule-containing composition (e.g., PCL NCs) can be used to deliver therapeutic agents to a targeted region of a subject having one or more doses contained in the delivered polymer-coated nanocapsules. In one embodiment, the therapeutic agent-containing polymer-coated nanocapsule composition can be similarly coated by one or more coating layers or include a mixture of polymer-coated nanocapsules of therapeutic agents where the outer coating layer of the polymer-coated nanocapsules varies in number of layers to produce a staggered release of therapeutic agents or a priming-like delivery followed by a boost delivery of at least one therapeutic agent. In certain embodiments, the priming dose of the at least one therapeutic agent can be the same or different than subsequent doses in the layered PCL nanocapsules.

In some embodiments and further to paragraphs [0064]-[0092] above, the polymer-coated nanocapsule-containing composition can be a single administration composition capable of treating, preventing or reducing the risk of onset of a health condition in a single dose. In certain embodiments, the single administration composition can include a single therapeutic agent or can include two, three, four or more different therapeutic agents embedded in the inner core. In other embodiments, when the two or more different therapeutic agents are present, the two or more different therapeutic agents can be contained together in a single representative polymer-coated nanocapsule having a single selected number of outer coating layers (e.g., one layer). In other embodiments, when the two or more different therapeutic agents are present, the two or more different therapeutic agents can be contained in two or more different representative polymer-coated nanocapsules having two or more different number of outer coating layers for delayed or varied timed delivery of the two or more different therapeutic agents. In certain embodiments, a single therapeutic agent can be encapsulated in polymer-coated nanocapsules disclosed herein having the same number of outer coating layers or different numbers of outer coating layers for delayed or varied timed delivery of the single therapeutic agents.

Other embodiments, and further to paragraphs [0064]-[0093] above, provide for methods for eliciting a response in a subject to the one or more therapeutic agents encapsulated in one or more polymer-coated nanocapsules, where the method can include administering a pharmaceutical composition described herein to the subject. In accordance with these embodiments, the pharmaceutical composition containing the one or more polymer-coated nanocapsules can be introduced to a subject and target a particular region or predetermined cell population or tumor of the subject and treat the subject.

Yet other embodiments provide for kits that can include at least one polymer-coated nanocapsule described herein and at least one container. In other embodiments, kits disclosed herein can include components for administering one or more types of polymer-coated nanocapsules to subject such as a health provider or caregiver.

### Type 1 Diabetes (TID)

In certain embodiments disclosed herein, designer polymer-coated nanocapsules having targeting peptides associated with at least one positively charged surfactant can be developed to overcome biological barriers and achieve cell-specific or other specific targeted region to deliver drugs. In certain embodiments, b-cells can be targeted to treat Type 1 diabetes. Type 1 diabetes (TID) is characterized by immune-mediated destruction of insulin producing β-cells located in the Islets of Langerhans in the pancreas. Current TID treatments use exogenous insulin to manage symptoms, but this is not a cure and the subject of TID must remain on exogenous insulin to survive. Some strategies to prevent β-cell stress or β-cell death or induce proliferation of remaining β-cells to produce endogenous insulin show promise in halting or reversing TID; however, lack of specific targeting to the β-cell by these methods has resulted in reduced efficacy and potential severe off-target effects. In certain embodiments, the polymer for this-cell targeted nanocapsule can include PCL and the one or more targeting agent can include a β-cell targeting agent. In accordance with these embodiments, PCL-coated β-cell targeted nanocapsules can be introduced to a subject having or suspected of developing type 1 diabetes and one or more therapeutic agents encapsulated within the PCL-coated β-cell targeted nanocapsules are released after the PCL-coated β-cell targeted nanocapsules are engulfed by β-cells of the subject to reduce, prevent or treat the type 1 diabetes in the subject by increasing survival and/or expansion of healthy insulin-producing β-cells in the subject or promoting the survival and engraftment of transplanted β-cells in the subject.

In other embodiments and further to paragraphs [0064]-[0096] above, targeted treatment using polymer-coated nanocapsules disclosed herein can be used to deliver at least one therapeutic agent capable of promoting the survival and engraftment of transplanted organs and/or cells in a subject receiving such a treatment.

In certain embodiments and further to paragraphs [0064]-[0097] above, polymer-coated nanocapsules disclosed herein can use less of the at least one therapeutic agent and reduce negative side effects than used by conventional delivery and provide enhanced efficacy after a single administration. In other embodiment, polymer-coated nanocapsules disclosed herein provide for thermostable formulations that eliminate and/or reduce refrigeration requirements (e.g., cold chain refrigeration requirements), limit the concentration or presence of adverse agents (e.g., preservatives etc.) administered to subjects, and increase efficacy due in part to targeted delivery of selected therapeutic agents. In other embodiments, compositions and methods disclosed herein are applicable to a variety of potential therapeutic uses as detailed herein.

In certain embodiments and further to paragraphs [0064]-[0098] above, a virus or virally-derived therapeutic agent can be, for example, a papovavirus (e.g., papillomaviruses, including human papilloma virus (HPV)), a herpesvirus (e.g., herpes simplex virus, varicella-zoster virus, bovine herpesvirus-1, cytomegalovirus), a poxvirus (e.g., smallpox virus), a reovirus (e.g., rotavirus), a parvovirus (e.g., parvovirus B19, canine parvovirus), a picornavirus (e.g., poliovirus, hepatitis A), a togavirus (e.g., rubella virus, alphaviruses such as chikungunya virus), a hepadnavirus (e.g., hepatitis B virus), a flavivirus (e.g., dengue virus, hepatitis C virus, West Nile virus, yellow fever virus, Zika virus, Japanese encephalitis virus), an orthomyxovirus (e.g., influenza A virus, influenza B virus, influenza C virus), a paramyxovirus (e.g., measles virus, mumps virus, respiratory syncytial virus, canine distemper virus, parainfluenza viruses), a rhabdovirus (e.g., rabies virus), a filovirus (e.g., Ebola virus), or a coronavirus or combinations thereof. In accordance with these embodiments, these therapeutic agents can include one or more therapeutic agents known in the art for treatment and/or prevention of an infection, for example or targeted treatment of an infected region of a subject such as the GI tract.

In other embodiments and further to paragraphs [0064]-[0099] above, a therapeutic agent disclosed herein can include a whole organism or a segment or fragment derived from a bacterium, a bacterial-derived agent, a bacteriophage for directed killing of a bacterial infection, or a toxin of a bacterium, including but not limited to, Pasteurella haemolytica, Clostridium difficile, Clostridium haemolyticum, Clostridium tetani, Corynebacterium diphtheria, Neorickettsia resticii, Streptococcus equi, Streptococcus pneumoniae, Salmonella spp., Chlamydia trachomatis, Bacillus anthracis, Yersinia spp., and Clostridium botulinum or combinations thereof.

In other embodiments and further to paragraphs [0064]-[0099] above, a therapeutic agent disclosed herein can include a whole organism or a segment or fragment derived from a fungus or an anti-fungal agent, including but not limited to, Cryptococcus spp. (e.g., neoformans and gatti), Aspergillus spp. (e.g., fumigatus), Blastomyces spp. (e.g., dermatitidis), Candida albicans, Paracoccidioides spp. (e.g., brasiliensis), Sporothrix spp. (e.g., schenkii and brasiliensis), Histoplasma capsulatum, Pneumocystis jirovecii and Coccidioides immitis, or combinations thereof.

In other embodiments and further to paragraphs [0064]-[0099] above, polymer-coated therapeutic agent-containing nanocapsules disclosed herein can include, at least one therapeutic agent to treat an animal such as a household pet, livestock, a horse, a bird, reptile or other animal. In accordance with these embodiments, the polymer-coated nanocapsules can be administered, for example, to a dog (canine), a cat (feline), a horse (equine), cattle (bovine), a goat (hircine), a sheep (caprine), or poultry (e.g., chicken, turkey, duck, goose).

In other embodiments and further to paragraphs [0064]-[0099] above, polymer-coated therapeutic agent-containing nanocapsule disclosed herein can be used to treat a canine to reduce onset of or prevent an infection or cancer or other condition. In accordance with these embodiments, treatments include but are not limited to, infections related to canine parvovirus (CPV), canine distemper virus (CDV), canine adenovirus (CAV), rabies, canine parainfluenza virus (CPiV), canine influenza virus, canine corona virus, measles virus, Bordetella bronchiseptica, Leptospira spp., and Borrelia burgdorferi or combinations thereof.

In other embodiments and further to paragraphs [0064]-[0099] above, polymer-coated therapeutic agent-containing nanocapsule disclosed herein can be used to treat a feline to reduce onset of or prevent an infection or cancer or other condition. In accordance with these embodiments, treatments include but are not limited to, infections related to immunogenic compositions directed to feline herpesvirus 1 (FHV1), feline calicivirus (FCV), feline panleukopenia virus (FPV), rabies, feline leukemia virus (FeLV), feline immunodeficiency virus, virulent systemic feline calicivirus, Chlamydophila felis, Pasteurella haemolytica, and Bordetella bronchiseptica or combinations thereof.

In other embodiments and further to paragraphs [0064]-[0099] above, polymer-coated therapeutic agent-containing nanocapsule disclosed herein can be used to treat an equine to reduce onset of or prevent an infection or cancer or other condition. In accordance with these embodiments, treatments include but are not limited to, infections related to immunogenic compositions directed to Eastern equine encephalomyelitis virus, Western equine encephalomyelitis virus, Venezuelan equine encephalomyelitis virus, bovine papillomavirus, rabies virus, Clostridium tetani, West Nile virus, equine influenza virus, Potomac fever (Neorickettsia risticii), Streptococcus equi, and rhinopneumonitis (equine herpesvirus type 1) or combinations thereof.

In other embodiments and further to paragraphs [0064]-[0099] above, polymer-coated therapeutic agent-containing nanocapsule disclosed herein can be used to treat a bovine to reduce onset of or prevent an infection or cancer or other condition. In accordance with these embodiments, treatments include but are not limited to, infections related to immunogenic compositions directed to bovine rhinotracheitis (IBR), parainfluenza type 3 (PI3), bovine virus diarrhea (BVD), bovine respiratory syncytial virus (BRSV), blackleg (Clostridium chauvoei), malignant edema (Clostridium septicum), infectious necrotic hepatitis (Clostridium novyi), enterotoxemia (Clostridium perfringens type C and D), Pasteurella haemolytica, and redwater (Clostridium haemolyticum) or combinations thereof.

In other embodiments and further to paragraphs [0064]-[0099] above, polymer-coated therapeutic agent-containing nanocapsules disclosed herein can be used to treat a human of any age to reduce onset of, or prevent an infection or cancer or other condition. In accordance with these embodiments, treatments include but are not limited to, treating infections in an infant or child or adolescent, including but not limited to, varicella-zoster (chicken pox), diphtheria, Haemophilus influenzae type b (Hib), hepatitis A, hepatitis B, influenza, measles, mumps, pertussis, polio, pneumococcal disease, rotavirus, rubella, and tetanus. In other embodiments, polymer-coated therapeutic agent-containing nanocapsules disclosed herein can be used to deliver one or more therapeutic or immunogenic agents to an adult, infant, pre-teen or teen, including but not limited to immunogenic agents against influenza, tetanus, diphtheria, pertussis, human papillomavirus, meningococcal disease, hepatitis B, hepatitis A, polio, measles, mumps, rubella, flavivirus or alphavirus-related condition, Ebola virus-related condition, human immunodeficiency virus, human papilloma virus, corona virus, SARS, and varicella-zoster.

Pathogenic agents and antigens derived therefrom contemplated herein can be in the form of recombinant peptide or protein immunogens, virus-like particles (VLPs), or inactivated or attenuated pathogens (e.g., viruses) or chimeric viruses or chimeric viruses in the same virus family such as flaviviruses, alphaviruses or the like. In certain embodiments, human papilloma virus (HPV) capsomeres can be incorporated into thermostable glassy microparticle via lyophilization without effect on the morphology of the HPV capsomeres.

In other embodiments, virus-like particles (VLPs) can be lyophilized into thermostable particles. VLPs resemble viruses, but do not replicate and contain viral genetic material. Therefore, VLPs have been demonstrated to be useful in vaccine formulations, providing a safer alternative to attenuated viruses. They contain high density displays of viral surface proteins that present viral epitopes that can elicit strong immune responses. In certain embodiments, VLPs from pathogens other than HPV can be incorporated into particles, similarly to HPV 16L1 VLP. Some non-limiting examples include VLPs of Hepatitis B, chikungunya virus, and influenza virus.

In yet other embodiments, inactivated or attenuated pathogens (e.g., live, attenuated viruses) can be lyophilized into thermostable microparticles. In accordance with these embodiments, inactivated (or killed) viruses or virus particles, bacteria, or other pathogens may be inactivated by any means, for example, chemically or by heat and incorporated into microparticles. Non-limiting examples of inactivated pathogens that may be incorporated into microparticles can include inactivated whole-cell pertussis (inactivated Bordetella pertussis), Salmonella typhi, and inactivated polio virus. Live, attenuated viruses or bacteria may similarly be incorporated into microparticles. Non-limiting examples of attenuated viruses and bacteria that may be incorporated into microparticles can include measles virus, mumps virus, rubella virus, influenza virus, chicken pox virus, smallpox virus, polio virus, rotavirus, flaviviruses (e.g., dengue virus, yellow fever virus), rabies virus, typhoid virus, Mycobacterium bovis, Salmonella typhi, and Rickettsia spp.

In some embodiments and further to paragraphs [0064]-[0110] above, buffers of use for compositions disclosed or for stabilizing therapeutic agents in an inner core (e.g., when combining with the at least one fatty acid) of polymer-coated nanocapsules disclosed herein can include, but are not limited to, acetate, succinate, citrate, prolamine, histidine, borate, carbonate or phosphate buffer, or a combination thereof. In certain embodiments, a buffer can include one or more volatile salts that can include, but is not limited to, one or more of acetate, sodium succinate, potassium succinate, citrate, prolamine, arginine, glycine, histidine, borate, sodium phosphate, potassium phosphate, ammonium acetate, ammonium formate, ammonium carbonate, ammonium bicarbonate, tricthylammonium acetate, triethylammonium formate, tricthylammonium carbonate, trimethylamine acetate trimethylamine formate, trimethylamine carbonate, pyridinal acetate and pyridinal formate, or combinations thereof. In certain embodiments, the buffer can include histidine, for example, histidine-HCl.

In some embodiments, glass-forming agents or sugar alcohols of use herein to coat or stabilize the NCs disclosed herein during storage or reconstitution processes can include, but is not limited to, trehalose, sucrose, ficoll, dextran, maltotriose, lactose, mannitol, hydroxyethyl starch, glycine, cyclodextrin, and povidone, or combinations thereof. In certain embodiments, a sugar alcohol can include mannitol, alone or in combination with another preservative and/or other sugar alcohol. In other embodiment, the mannitol concentration can be present in a weight-to-volume (w/v) concentration from about 0.1% to about 40% in a composition prior to creating a nanocapsule; from about 1% to about 30% w/v; from about 0.5% to about 20%; or from about 1.0% to about 15% w/v in the composition. In another embodiment, the glass-forming agent can be mannitol or trehalose or combination thereof in a concentration from about 1.0% to about 15%; or about 5.0% w/v.

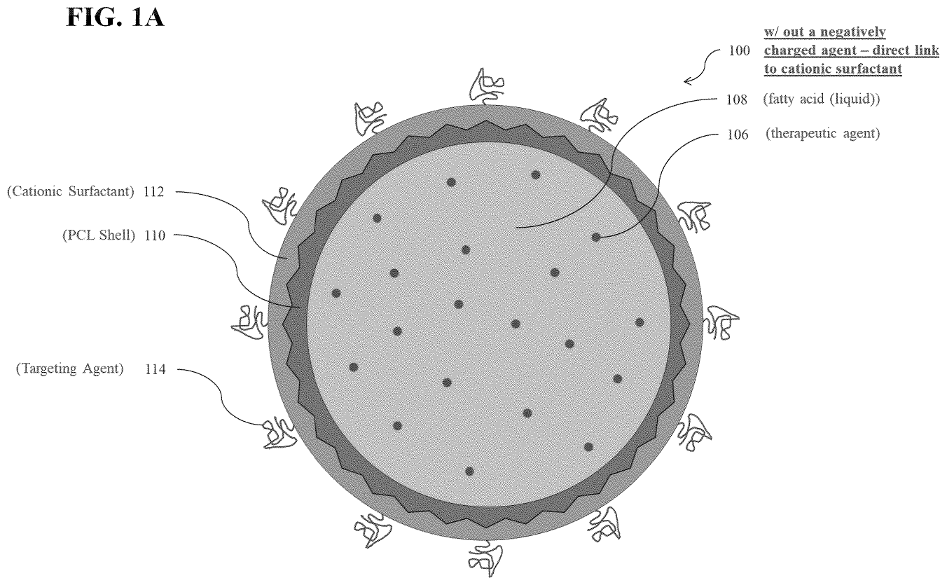

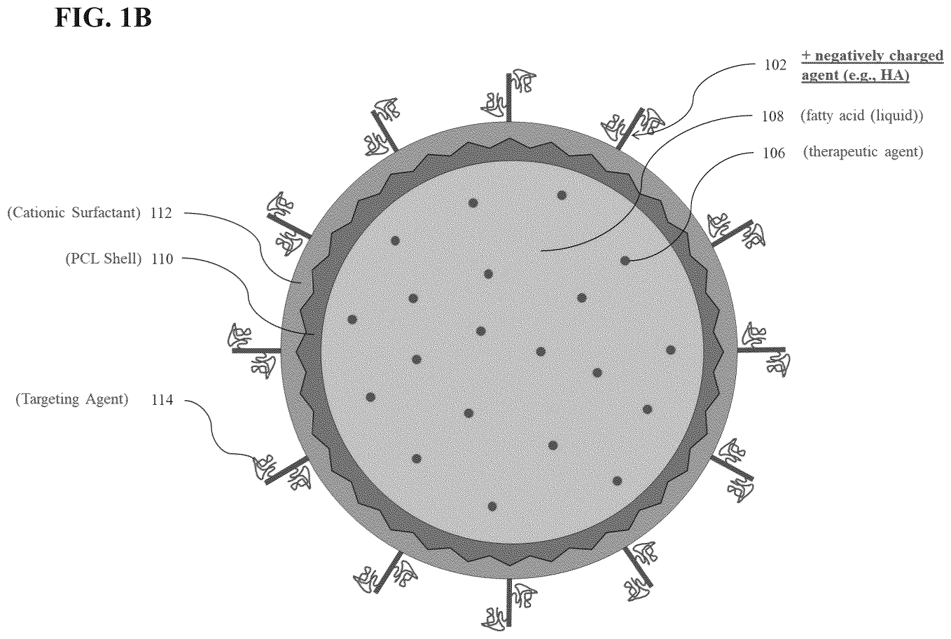

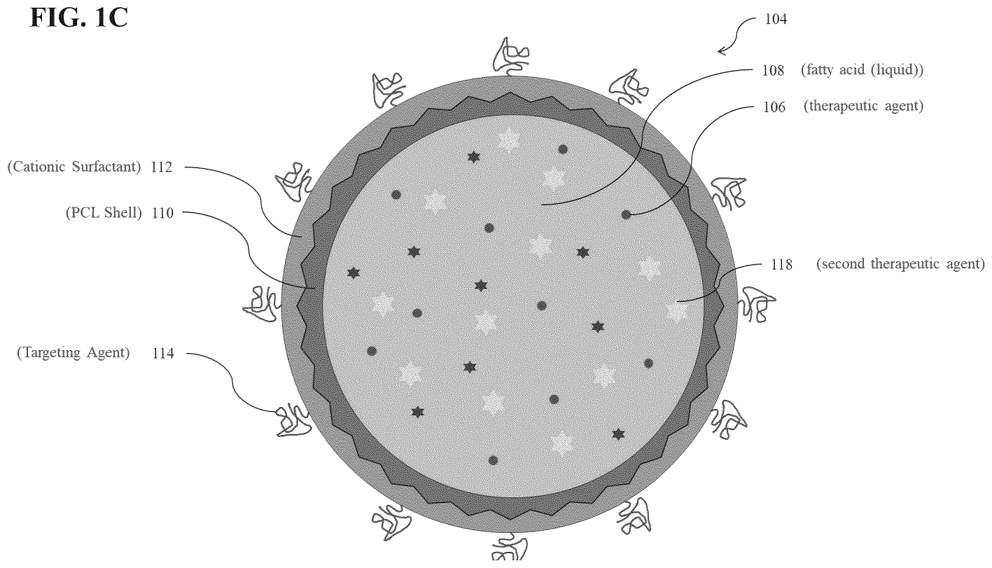

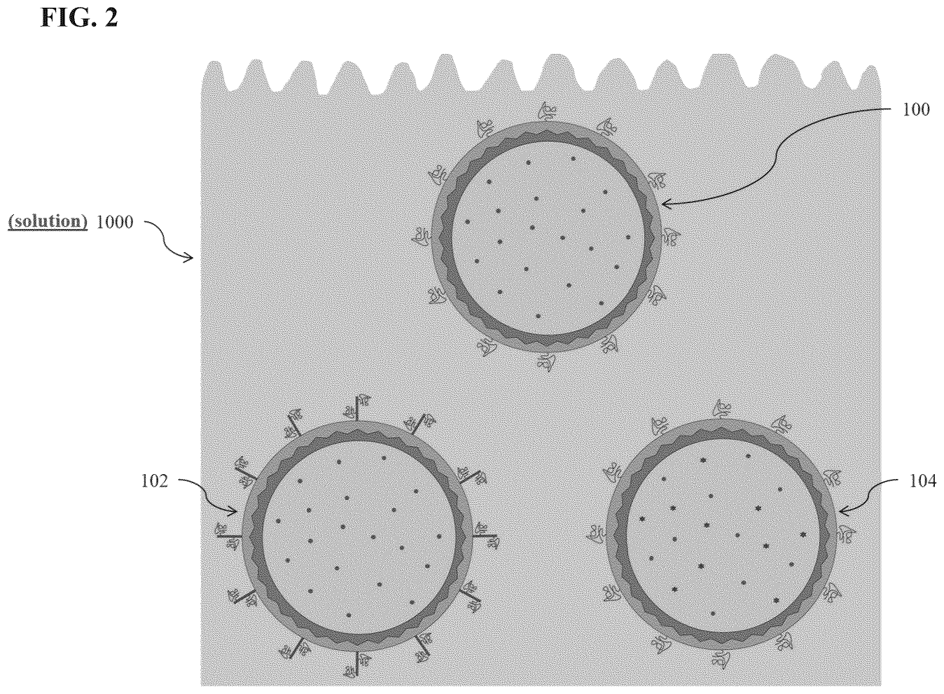

In certain embodiments, exemplary polymer-coated nanocapsules of the present invention can be as schematically represented as in FIGS. 1A-1C. These diagrams illustrate certain polymer-coated nanocapsules contemplated of used herein. As illustrated in the example of FIG. 1A, polymer-coated nanocapsules 100 can include at least one fatty acid 108 and at least one therapeutic agent 106 forming an inner core. The inner core coated by at least one polymer to form a polymer shell 110. The polymer shell can further include at least one cationic surfactant 112 and at least one targeting agent 114. As illustrated in the example of FIG. 1B, in certain embodiments, polymer-coated nanocapsules 120 can include at least one fatty acid 108 and at least one therapeutic agent 106 forming an inner core. The inner core coated by at least one polymer to form a polymer shell 110. The polymer shell can further include at least one cationic surfactant 112 and at least one targeting agent 114 where the targeting agent is linked to the cationic surfactant through a second molecule, a negatively charged agent, 102. As illustrated in the example of FIG. 1C, in certain embodiments, polymer-coated nanocapsules 130 can include at least one fatty acid 108 and at least one therapeutic agent 106 forming an inner core. The inner core coated by at least one polymer to form a polymer shell 110. The polymer shell can further include at least one cationic surfactant 112 and at least one targeting agent 114 and further include a second therapeutic agent, 118. In certain embodiments, polymer-coated nanocapsules disclosed herein can be stored in aqueous, or in essentially dry form or rehydrated after being essentially dried for prolonged storage and later use. A single type or multiple types of polymer-coated nanocapsules disclosed herein can be stored separately and optionally, later mixed for delivery or stored in combination compositions for ready use. In other embodiments, a mixture of these nanocapsules can be reconstituted into a single-administration composition. In other embodiments, a prime therapeutic agent dose can be administered to a subject followed by a boost or a prime and at least one boost dose can be provided by single or multiple administrations. In another embodiment, FIG. 2 illustrates a mixed composition of several different polymer-coated nanocapsules in solution, 1000.

### EXAMPLES

The materials, methods, and embodiments described herein are further defined in the following Examples. Certain embodiments are defined in the Examples herein. It is understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

### Example 1

### Generation of Polymer-Coated Nanocapsules for Therapeutic Agent Delivery

In certain exemplary methods, nanocapsules can be created using a PCL polymer shell with a charged surfactant (e.g., BKC or other appropriate surfactant) contained in the pores of the polymer shell having an oil-containing inner core with one or more therapeutic agents encapsulated inside. The charge from the cationic surfactant (e.g., BKC) allows for example, peptides or full-length proteins to adsorb onto the surface of the nanocapsules via electrostatic interactions. This facilitates having any peptide or protein or other molecule attached to the surface. In one example, a formulation includes an antibody or peptide tethered to a low molecular weight negatively charged agent (e.g., hyaluronic acid (HA)) coating on the outside of the nanocapsules. However, this technology is compatible with many ionic molecules, including peptides or other ionic molecules. Further, the fatty acid core allows encapsulation of a large variety of therapeutics, including, but not limited to, RNA, DNA, peptides, and small molecules.

In certain examples, exemplary polymer-coated nanocapsules of the present invention can be as schematically represented as in FIGS. 1A-1C. These diagrams illustrate certain polymer-coated nanocapsules contemplated of used herein. As illustrated in the example of FIG. 1A, polymer-coated nanocapsules 100 can include at least one fatty acid 108 and at least one therapeutic agent 106 forming an inner core. The inner core coated by at least one polymer to form a polymer shell 110. The polymer shell can further include at least one cationic surfactant 112 and at least one targeting agent 114. As illustrated in the example of FIG. 1B, in certain embodiments, polymer-coated nanocapsules 120 can include at least one fatty acid 108 and at least one therapeutic agent 106 forming an inner core. The inner core coated by at least one polymer to form a polymer shell 110. The polymer shell can further include at least one cationic surfactant 112 and at least one targeting agent 114 where the targeting agent is linked to the cationic surfactant through a second molecule, a negatively charged agent, 102. As illustrated in the example of FIG. 1C, in certain examples, polymer-coated nanocapsules 130 can include at least one fatty acid 108 and at least one therapeutic agent 106 forming an inner core. The inner core coated by at least one polymer to form a polymer shell 110. The polymer shell can further include at least one cationic surfactant 112 and at least one targeting agent 114 and further include a second therapeutic agent, 118. In certain embodiments, polymer-coated nanocapsules disclosed herein can be stored in aqueous, or in essentially dry form or rehydrated after being essentially dried for prolonged storage and later use. A single type or multiple types of polymer-coated nanocapsules disclosed herein can be stored separately and optionally, later mixed for delivery or stored in combination compositions for ready use. In other embodiments, a mixture of these nanocapsules can be reconstituted into a single-administration composition. In other examples, a prime therapeutic agent dose can be administered to a subject followed by a boost or a prime and at least one boost dose can be provided by single or multiple administrations. In another example, FIG. 2 illustrates a mixed composition of several different polymer-coated nanocapsules in solution, 1000 where each polymer coated nanocapsule can contain a single or multiple therapeutic agents and/or where each polymer coated nanocapsule can contain the same or different therapeutic agents and/or where each polymer coated nanocapsule can contain hydrophobic or hydrophilic therapeutic agents.

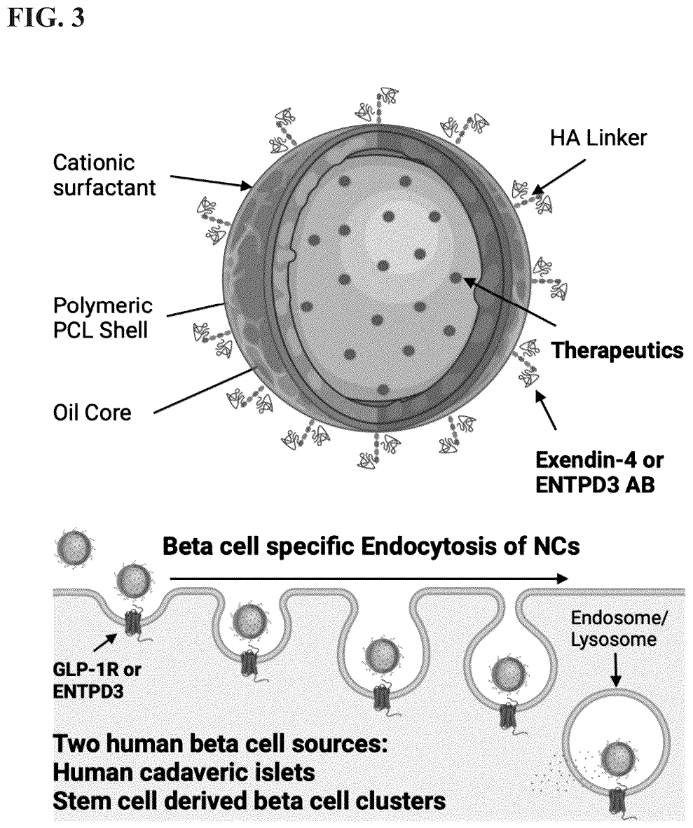

FIG. 3 illustrates a schematic diagram demonstrating certain components of an exemplary polymer coated nanocapsule disclosed herein and engulfment of a polymer-coated nanocapsule by a targeted cell (or tissue) when the polymer-coated nanocapsule reaches its target and is endocytosed. In addition, dispersion of the therapeutic agent occurs after engulfment of the polymer coated nanocapsule by the targeted cell (or tissue).

### Generation of Polymer-Coated Nanocapsules for Hydrophobic Cargo

In one exemplary method, PCL-NCs were synthesized using a water in oil (W/O) emulsion technique for encapsulating hydrophobic cargo (e.g., hydrophobic therapeutic agent). A schematic of this process is depicted in FIG. 5A. In this example, the oil phase includes two solutions. The first solution is coconut oil (60 μl) dissolved in ethanol (750 μl) and the second solution is PCL (2 mg/ml) with the addition of a coating, e.g., BKC (20 wt/wt % PCL) dissolved in acetone (4.25 ml). Both solutions were combined and stirred briefly and then poured into the aqueous phase, including about 10 mL deionized water followed by 1-2 min of rigorous stirring. The ethanol and acetone were removed using a rotavapor (R-100, Buchi, New Castle, DE), leaving a NC suspension in DI water. NCs were washed twice by centrifugation at 20,000 rcf for 10 min and resuspending the NCs in fresh DI water. W/O synthesized BKC-PCL-NCs were loaded with 10 μl of either BODIPY or Cy5 (as an example of a therapeutic agent) dissolved in the ethanol solution and the volume of cargo added was subtracted from the amount of ethanol added allowing the total volume to remain at 750 μl. To form coated NCs, the W/Os NCs were then incubated as a coating either with bovine serum albumin (BSA) or hyaluronic acid (HA) for at least 30 min at room temperature and then washed via centrifugation as previously stated above. Multiple other suitable cargos can be introduced to these nanocapsules. Representative TEM and SEM images of blank (no cargo) W/Os are illustrated in FIG. 5B-5C, respectively. A representative confocal image of W/O NCs loaded with BODIPY is illustrated in FIG. 5D, with BODIPY particles visible in green due to fluorescence.

### Generation of Polymer-Coated Nanocapsules for Hydrophilic Cargo

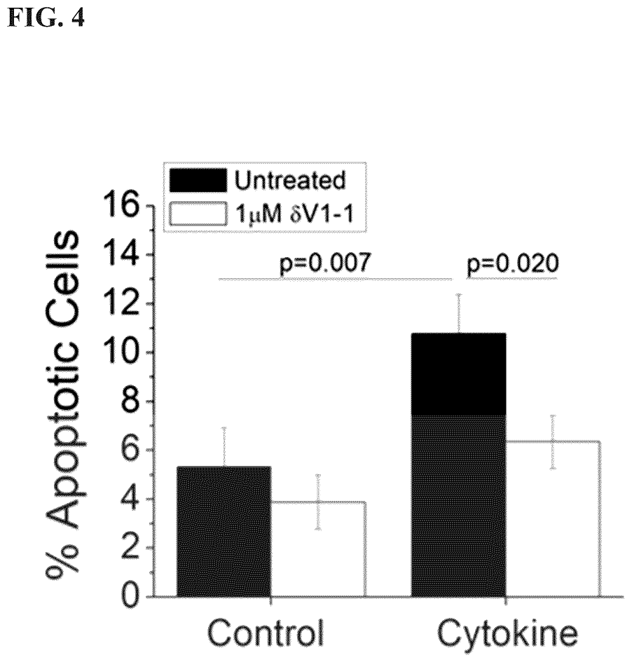

In another exemplary method, a water in oil in water (W/O/W) double emulsion-solvent evaporation synthesis was used to encapsulate hydrophilic cargo in NCs (e.g., hydrophilic therapeutic agents). A schematic of this process is depicted in FIG. 6A. The primary emulsion (W1:O) phase included hydrophilic cargo dissolved in DI water (W1) and coconut oil (O) at a ratio of 40:60. The DI water with the dissolved cargo was combined with the coconut oil and sonicated (e.g., at about 20% power for 3 min on ice). The W:O emulsion was then combined with DI water (W) at a 1:10 ratio and sonicated (e.g., at 20% power for 3 min) (W/O/W). A solution containing PCL (2 mg/ml) and BKC (20 wt/wt % PCL) dissolved in 4.25 ml acetone was added dropwise to the W/O/W emulsion under vigorous stirring for about 2 min. The acetone was evaporated and the NCs were washed as described above. Then, coating the W/O/W NCs either with BSA or HA was the same as stated above. All parameters were tested in triplicate as the (W₁:O) NCs stated above. FIG. 6B-6C illustrate representative SEM images of uncoated (FIG. 6B) or BSA coated (FIG. 6C) blank (i.e., no cargo) W/O/W NCs. FIG. 6D illustrates representative confocal images of W/O/W NCs loaded with a representative hydrophilic cargo, Rh123 (visualized in red by detection of the Rh123). Multiple other suitable cargos can be introduced to these nanocapsules. For example, another suitable cargo can include 8V1-1, a hydrophilic therapeutic peptide, which has been demonstrated to have a protective effect against cytokine toxicity in vitro (See for example, FIG. 4).

### Example 2

### Nanocapsule Characterization

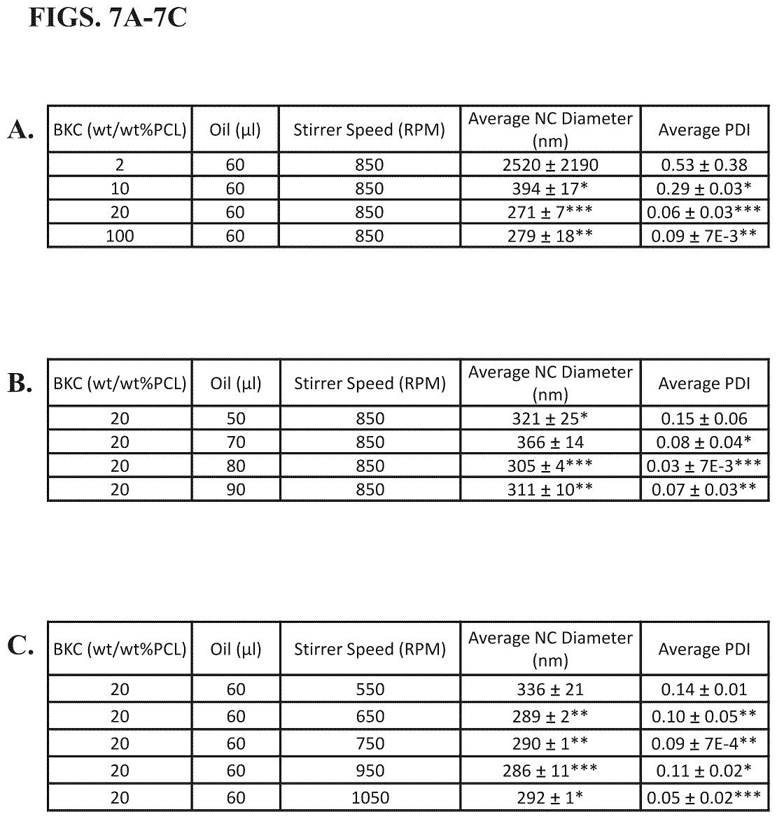

In another exemplary method, to determine if various parameters influence NC diameter, BKC concentrations in addition to other factors such as oil core volumes, and rate of stirring were altered to determine the optimal NC formulation (FIGS. 7A-7C). For the three parameters altered (e.g., coating, oil concentration and stirrer speed), BKC concentration had the largest variation in both NC size (sem±640 nm) and PDI (sem±0.12), followed by changes in oil volumes (sem±16 for NC size, sem±0.03 for PDI) and then stirring speeds (sem±12 for NC size, sem±0.02 for PDI) (FIGS. 7A-7C). Out of all combinations tested the sample chosen for the remainder of the Examples disclosed herein for the W/O emulsion included the following parameters: BKC (20 wt/wt % PCL), 60 μl oil, and 850 rpm stirring speed which yielded the smallest average NC diameter of 271±7 nm with an average PDI of 0.06±0.03, respectively (FIG. 7A). These exemplary results suggest that lower concentrations of BKC surfactant do not support an effective emulsion solution for efficient NC formation of uniform size and that a similar concentration dependence may be observed for similar cationic surfactants. It is noted that there was less than a 10 nm difference between the 20 wt/wt % and 100 wt/wt % PCL BKC coated NCs disclosed herein (FIG. 7A). This observed plateau suggests that the threshold of BKC embedded in the PCL polymer matrix had likely been reached when intertwined into the PCL mesh framework.

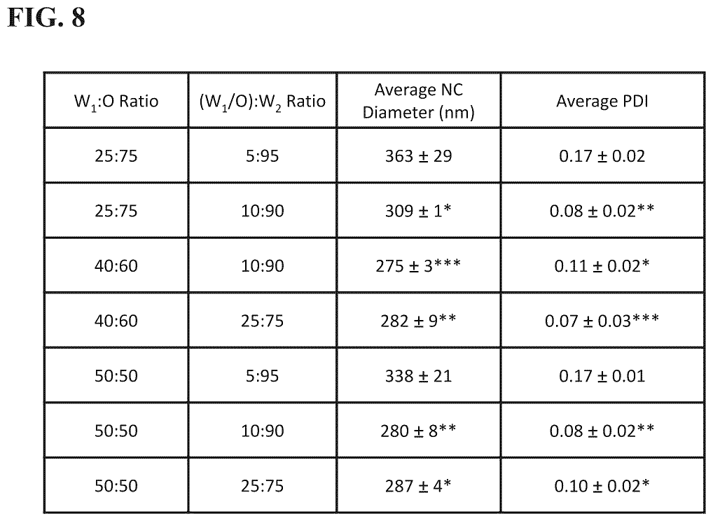

In another exemplary method, both W:O and (W₁:O)/W₂ ratios were altered for the W/O/W NCs, (See FIG. 8). The W1:O phase was tested at various ratios ranging from about 25:75-50:50 and ratios of the (W1:O)/W2 emulsion were tested at volume ratios from about 5:95-25:75. NCs formulated with a 25:75 W1:O and 10:90 (W1:O)/W2 had a significantly higher NC diameter from the other two treatments with a 10:90 (W1:O):W2 phase (p=0.03 40:60 W1:O and p=0.02 50:50 W1:O, FIG. 8). This suggests that one optimum ratio for the formation of NCs using the W/O/W method is about 40.0-50.0 ul W₁ to about 50.0-75.0 ul O and about 10.0-25.0 ul W₁/O to about 75.0-90.0 ul W₂ for generating uniformly sized NC.

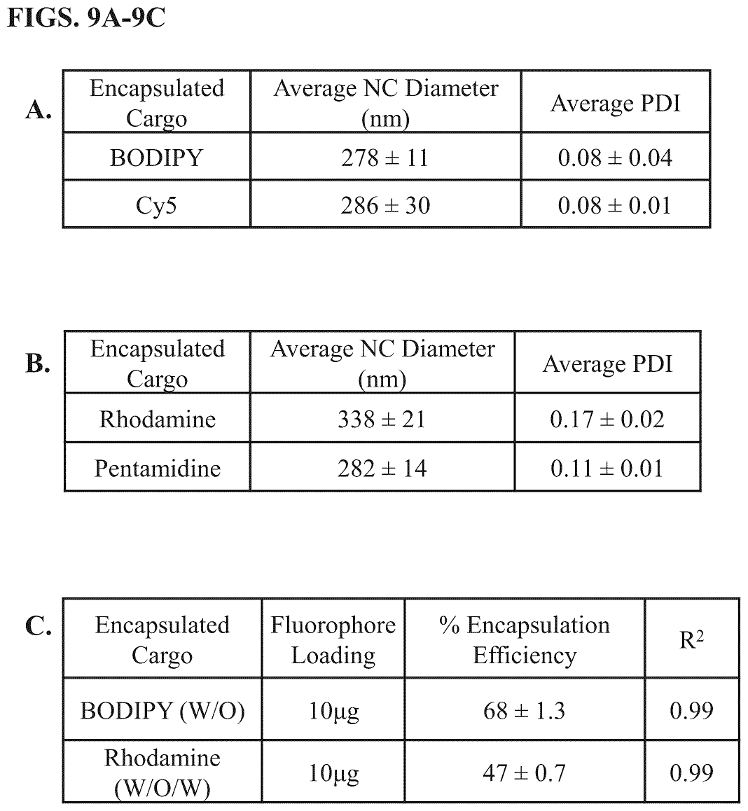

As noted above, the diameter and relative monodisperse size of the NCs synthesized by W/O and W/O/W were confirmed by SEM and TEM images (FIGS. 5B-5C and FIG. 6B). Successful encapsulation of hydrophobic cargo for W/O NCs (FIG. 5D) and hydrophilic cargo for W/O/W NCs (FIG. 6D) were confirmed by confocal microscopy. These results highlight that NCs disclosed herein can encapsulate and deliver cargo that typically has difficulty overcoming biological hurdles such as transporting hydrophilic therapeutics across the cell membrane. Congruent with DLS measurements, SEM and TEM also illustrated relatively homogenous size distributions for coated NCs (FIG. 6C). There was less than a 20 nm increase in NC diameter for the W/O NC diameter when encapsulating either BODIPY or Cy5 while maintaining homogenous size distributions (FIG. 9A) which is consistent with other studies. This was also observed with PTM loaded W/O/W NCs however, the Rh123 loaded W/O/W NCs had a 63 nm diameter increase as well as slight increase in PDI (FIG. 9B). The bulky structure of Rh123 could be attributing to the increase in NC size as another nanoparticle study demonstrated about a 50 nm increase when comparing Rh123 NPs compared to the unloaded control. Finally, the encapsulation efficiency (EE) of the BODIPY encapsulated W/O NCs was 68±1.3% and 47±0.7% for Rh123 encapsulated W/O/W NCs (FIG. 9C).

### Effect of Surface Coating on Size and Uniformity

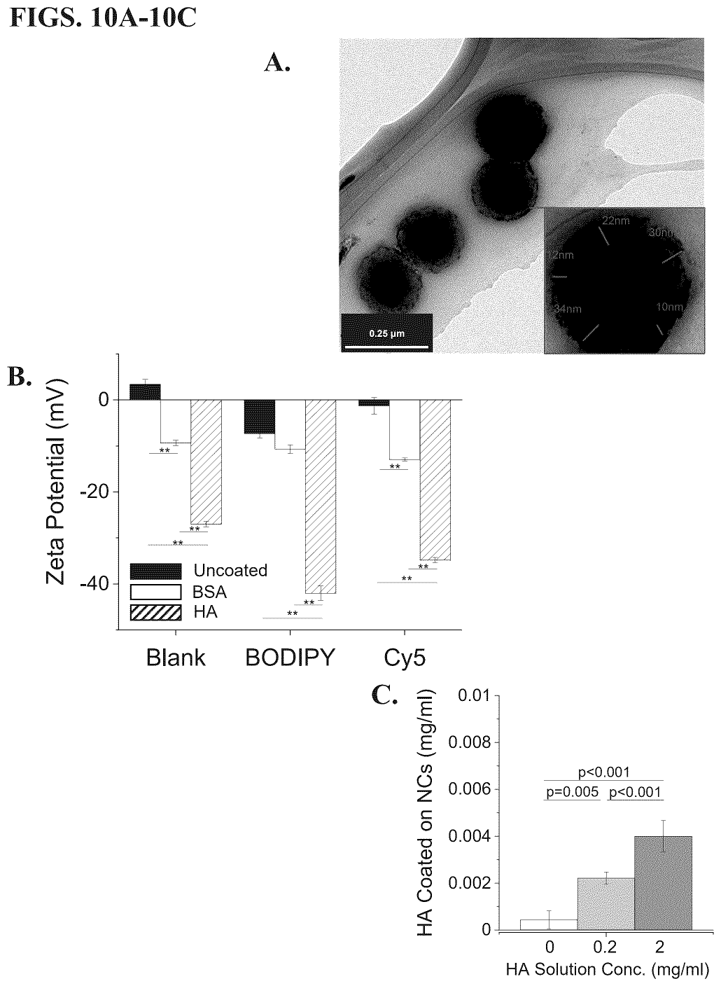

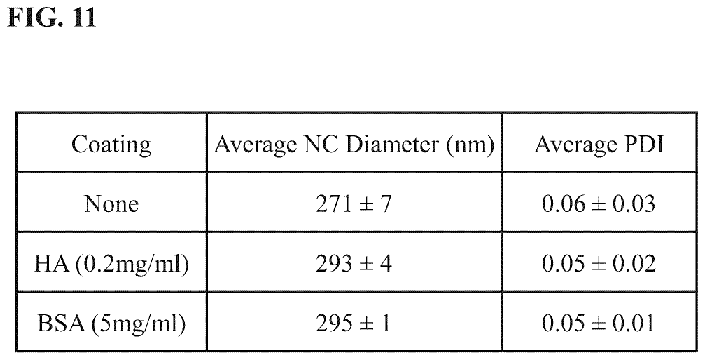

In another method, NCs were coated either with BSA or HA as described above and then measured with dynamic light scattering (DLS) to measure diameter and uniformity (PDI). Compared to the average uncoated W/O NC diameter of 271±7 nm, HA coated NCs had an average diameter of 293±4 nm and BSA of 295±1 nm (FIG. 11). This equates to an average diameter increase of 22 nm for HA and 24 nm for BSA coated W/O NCs. SEM and TEM images both support the DLS spectra (FIG. 5B, FIG. 10A). Furthermore, measurements were taken from the TEM images between the lighter shell and darker core from the longest and shortest widths which averaged between 20-30 nm supporting the increase in NC size displayed by the DLS (FIG. 10A, FIG. 11). All PDIs were under 0.1 and it was observed that there was only about a 0.01 difference between the coated and uncoated NCs demonstrating all formulations were monodispersed. The significance of being able to adhere various negatively charged coatings to the outside of the NCs is that this allows us to utilize different cell targeting moieties to increase cell specificity.

### Effect of Cargo on Surface Charge

In another exemplary method, W/O NC surface charge as measured by zeta potential was utilized to test if BSA and HA adhered to the outside of the NCs and if encapsulated cargo, BODIPY (anionic) and Cy5 (cationic) alters or significantly alters NC charge (FIG. 10B). Although Cy5 did have a slight increase in zeta potential it did not significantly alter the NC charge compared to the blank (cargo-less) W/O NCs (FIG. 10B). Although the zeta potential of the uncoated BODIPY NCs was generally as expected, there was a significant negative charge compared to both the uncoated blank (p<0.001) and uncoated Cy5 W/O NCs (p=0.013, FIG. 10B). When comparing the uncoated to both HA and BSA coated W/O NCs, all conditions tested except the blank BSA coated and BODIPY loaded BSA coated W/O NCs had a significant negative charge (p<0.001) ranging from −9-−12 mV for BSA and −27-−42 mV for HA (FIG. 10B). Furthermore, only blank W/O NCs had a significant negative charge (p<0.01) using 0.2 mg/ml HA and 5 mg/ml HA coated NCs compared to the W/O NCs containing encapsulated cargo (FIG. 12A). In addition, when comparing different BSA concentrations (2.5 mg/ml, 5 mg/ml, and 10 mg/ml), the BODIPY encapsulated W/O NCs with 10 mg/ml BSA had a significant negative charge compared to the 5 mg/ml (p<0.001) and 2.5 mg/ml (p<0.001, FIG. 12B). These results indicate that negatively charged surface coatings can be applied to the NCs over a range of approximately 2.5-10.0 mg/ml as a method to attach targeting peptides or to modulate the surface charge of the NC for directed delivery. These results are critical as charge can influence important factors such as cytotoxicity, cell permeability, and efficacy. The ability to provide surface charge modifications on the NCs disclosed herein is an incredible advantage as this allows the conjugation of ligands, peptides, antibodies, etc. for improved cell or target specificity making these NCs a valuable therapeutic agent-containing delivery vehicle as well as a screening tool for cell specific or target specific moieties for biological applications.

Stability of Nanocapsules after Freeze Drying and/or Culturing

In another exemplary method, NC stability after freeze drying was tested as a potential method for preserving NCs for long term storage and use including analyzing NC integrity and cargo retention for extended periods of time. In this example, mannitol (a sugar alcohol example, e.g., sorbitol and the like) was chosen as the cryoprotectant. W/O NCs were utilized for this experiment. Mannitol concentrations of 2.5% w/v, 5.0 w/v, and 10.0 w/v were tested to determine the optimal concentration (FIG. 13A). It was observed that there were no significant increases in NC size before and after freeze drying under each mannitol condition (FIG. 13A). Untreated NCs did not survive the freeze-drying process demonstrating a cryoprotectant such as a sugar alcohol is important for freeze drying FIG. 13A). SEM images were taken before (FIG. 13B) and after (FIG. 13C) freeze drying to confirm DLS measurements. It was observed that the NC yield decreased after reconstitution which may be attributed to the mannitol crystallizing and imposing mechanical forces either breaking the NCs or forming aggregates. It is noted that mannitol can be removed, if needed or desired, from the NC formulations by centrifugation and washing in either water or PBS. In certain methods, sucrose, sucralose, and glucose were unable to replace mannitol in these freeze-drying processes as they failed to stabilize the NCs.

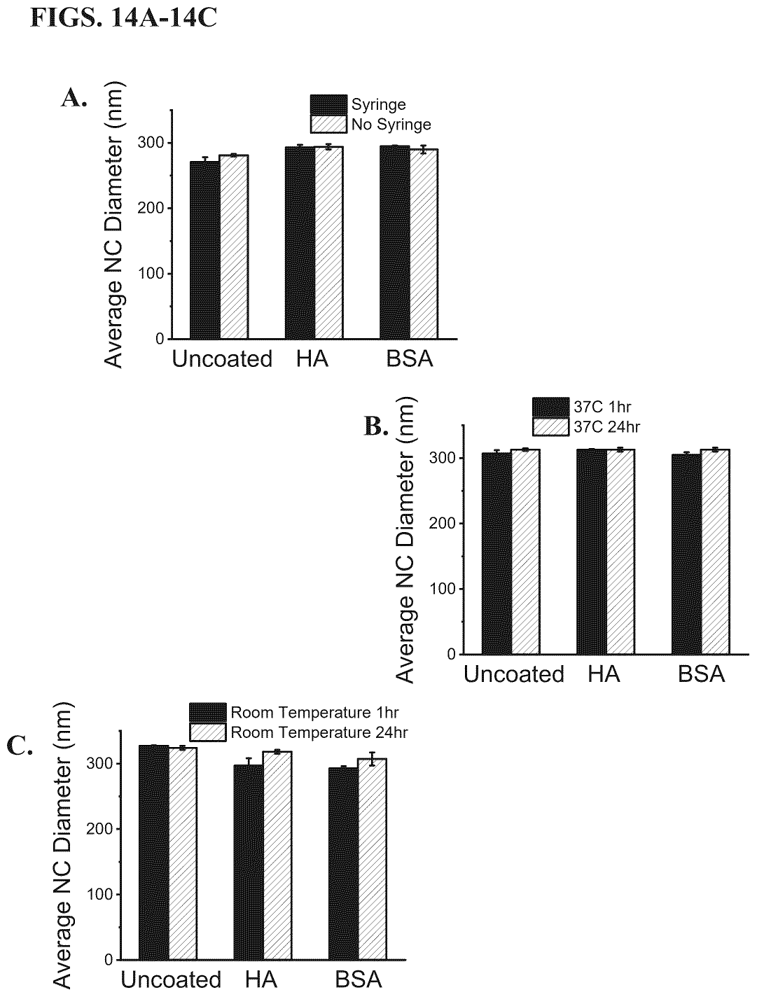

In another exemplary method, the stability of uncoated, HA coated, and BSA coated W/O NCs was tested in DMEM culture media in vitro as well as being extracted and passed through a 29G 1 cc insulin syringe. It was observed that there was no significant difference in NC size or PDI after passing NCs through a 29G syringe as well as being incubated in DMEM for 1 and 24 hour either at 37° C. or room temperature (FIGS. 14A-14C). All PDIs for all conditions were under 0.12. Overall, the characterization data demonstrates the feasibility, stability and interchangeable capabilities for both charge and encapsulated cargo of the NCs. This supports the disclosed NCs as having improved potential for cell targeted drug delivery applications.

### Example 3

### In Vitro Cargo Release of W/O and W/O/W NCs

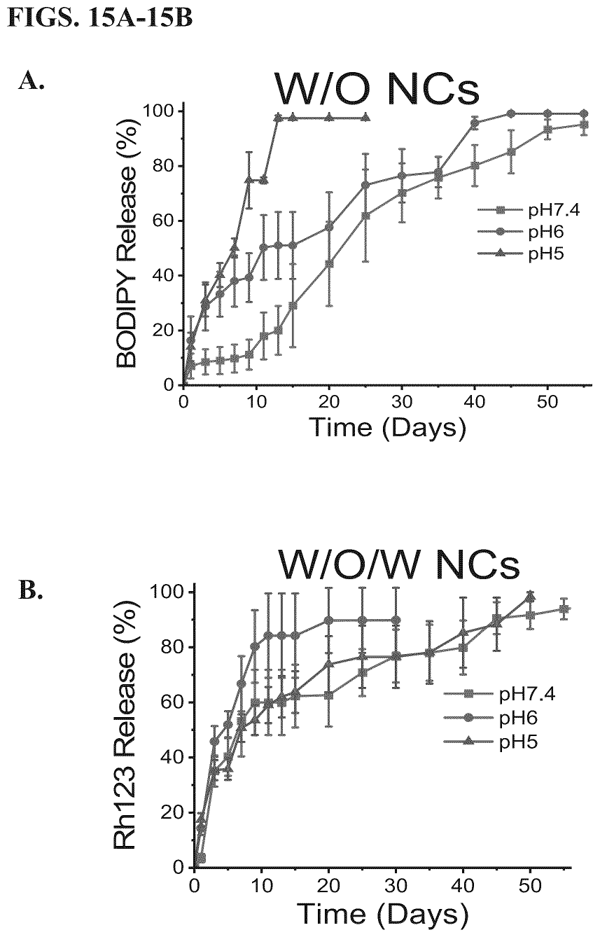

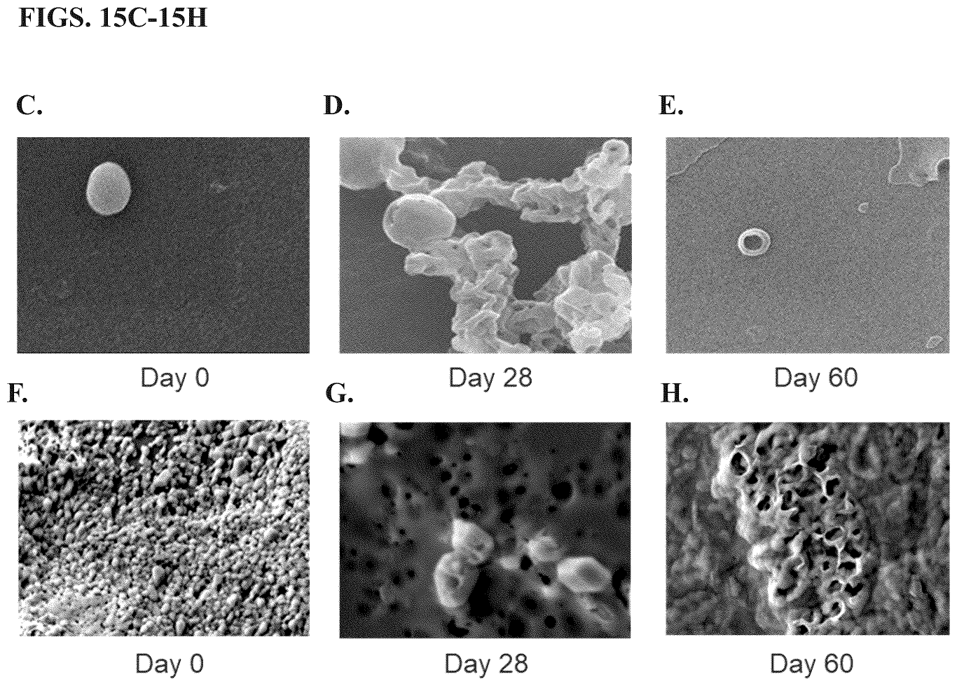

In another exemplary method, BODIPY (W/O, hydrophobic) and Rh123 (W/O/W, hydrophillic) loaded NCs were placed in 1×PBS reservoirs at varying pHs to assess release rates. For the BODIPY loaded W/O NCs the total average release time was about 45-50 days when incubating in PBS pH 7.4 (FIG. 15A). There was no significant difference for timepoints of about pH 7.4 and about pH 6.0. For pH 5.0 and pH 6.0 NCs, both pH conditions had a similar initial burst release in the first 24 hrs (FIG. 15A). Although pH 6 and pH 5 NCs have a higher initial burst release compared to pH 7.4, there was only a significant difference in release between NCs incubating in pH 7.4 versus pH 5 on day six (p=0.004) and day seven (p=0.004) in the first week (FIG. 15A). NCs in pH 5.0 had close to 100% of the cargo released by the end of the second week (FIG. 15A). Furthermore, pH 5.0 had a significantly faster release profile compared to pH 7.4 every day during the second week (p=0.12, p<0.001, p=0.001, p=0.002, p=0.004, p<0.001, p=0.002) and only on the 13th (p=0.04) and 14th day (p=0.04) compared to pH 6. There was no significant change for any timepoint in the release profiles for Rh123 loaded W/O/W NCs (FIG. 15B). These results provide support that pH influences the degradation and in turn the release profiles of the NCs. The faster degradation rate at lower pH is likely caused by the increase in hydrogen ions leading to increased hydrolytic degradation by random chain fission as this has been demonstrated as a major contributor in PCL degradation. Understanding this pH dependent mechanism is imperative because if the NCs are successfully endocytosed, they may be transported to cellular organelles such as lysosomes or endosomes which have lower pH values (pH about 4-6).

In another method, SEM images were taken on day 0, 30, and 60 to observe the degradation morphology of both the W/O and W/O/W NCs (FIGS. 15C-15H). By day 30, pores begin to appear on the NC surface (FIG. 15D, FIG. 15G) supporting the release of fluorescent cargo as illustrated by the release studies (FIG. 15A and FIG. 15B). By day 60, it appeared that all cargo had been released (FIG. 15A and FIG. 15B) and only a partial PCL shell remains (FIG. 15E and FIG. 15H).

### Example 4

Delivery of NCs into Human Islet Cells

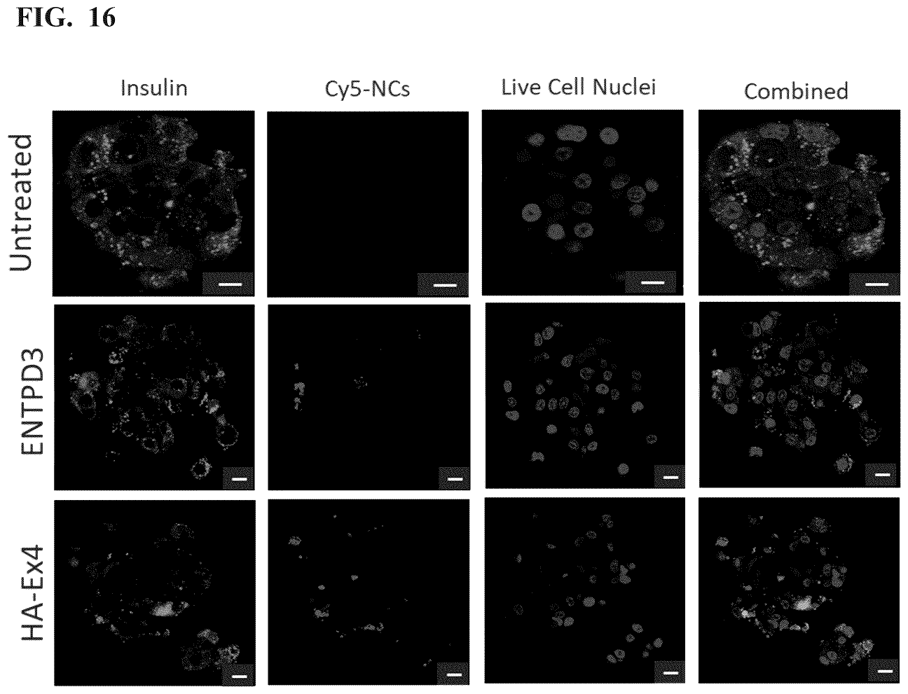

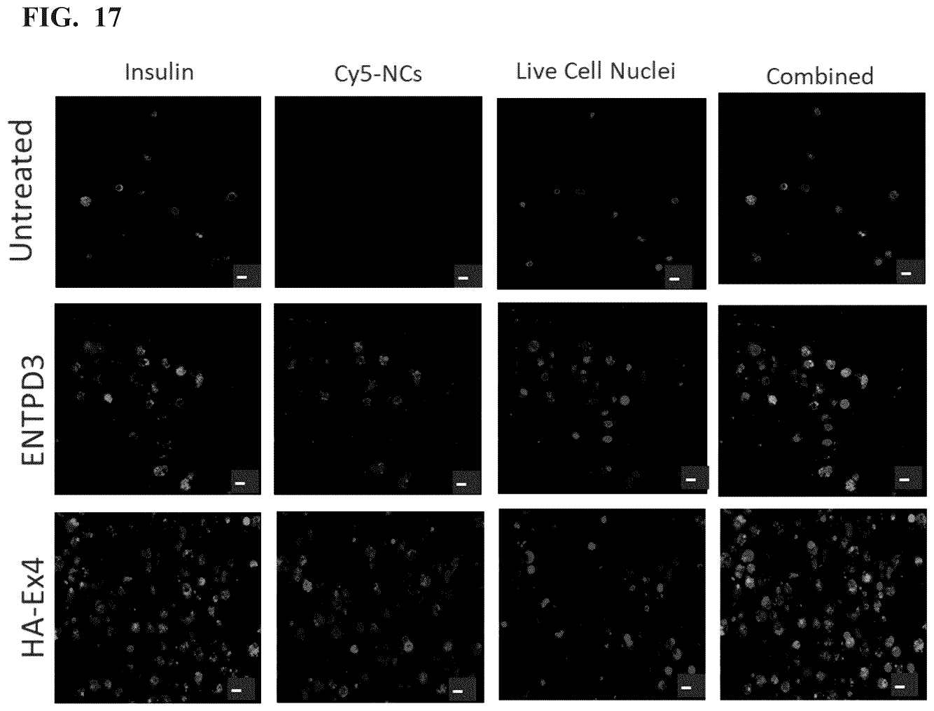

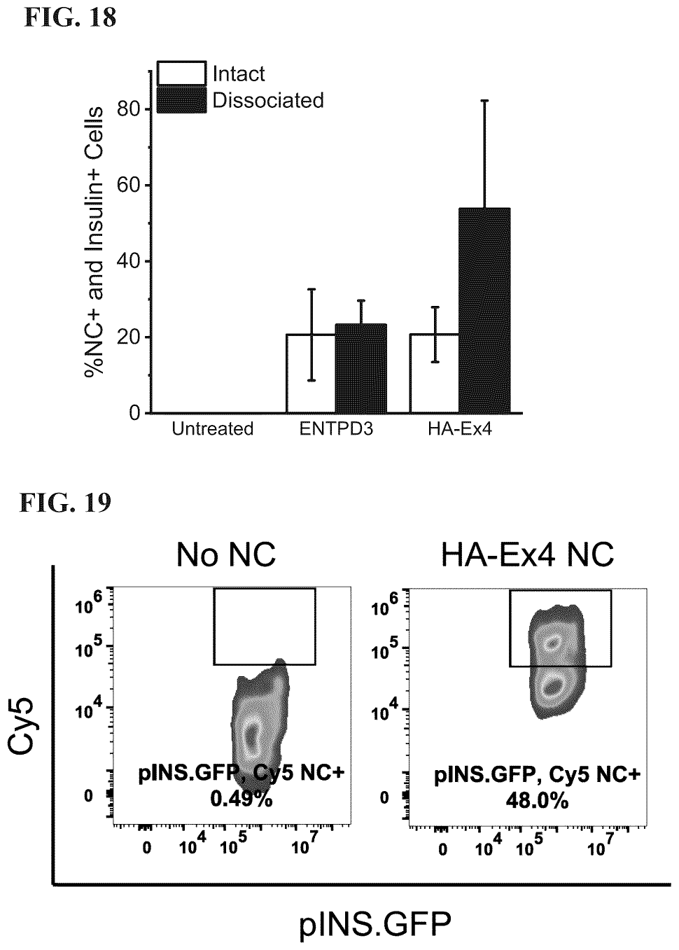

To determine if the NCs could be taken up by islet cells, human cadaveric islets were used. NCs loaded with Cy5 and coated with either ENTPD3 antibody or HA-Ex4 were incubated with human islets, either intact or dissociated into single cells for 24 hours. Islets were stained with FluoZin-3, a zinc sensor that specifically labels islet β-cells. By 24 hours the ENTPD3 coated and HA-Ex4 coated NCs were endocytosed into the islet as illustrated by the red fluorescence (second column) compared to the untreated control (FIG. 16). This demonstrates that NCs with both targeting peptides can target and be taken up by human β-cells in vitro. In dissociated human islets, an increase in NC uptake is observed for cells treated with HA-Ex4 coated or ENTPD3 coated NCs (FIG. 17). This demonstrates that NC uptake increases as access to the cell surface is increased through dissociation. Additionally, NC uptake was quantified in β-cells in both intact and dissociated human islets (FIG. 18) and found NC enrichment in ˜20% of β-cells in intact islets and ˜20-50% of dissociated β-cells after 24-hour treatment. Overall, this data supports that in vivo delivery of NCs supports improved NC uptake compared to in vitro isolated islet experiments as more of the islet surface will be available for uptake through the vasculature compared to in vitro where only the outer surface of the islet is available for NC uptake.

In some exemplary methods, stem cell derived β-cells (referred herein as “sBCs”) can be used to further test nanocapsule uptake in a physiologically relevant system. These cells are described in Santini-Gonzalez et al., (Front. Endocrinol., Sec. Diabetes: Molecular Mechanisms Volume 13-2022) which is incorporated herein by reference in its entirety. These cells are reporter sBCs containing a GFP reporter gene under the control of the endogenous insulin promoter and a constitutive firefly luciferase gene allowing convenient visualization and live in vivo quantification. The sBCs also behave as expected, responding appropriately to glucose and potassium stimulation in the presence or absence of other augmenting factors (such as Exendin-4, which augments glucose response in β-cells). Further, as illustrated in this reference, they can be transduced into mouse model where they still function as normal β-cells—allowing for in vivo delivery of pharmaceutical agents using the exemplary nanocapsules of the present disclosure.

In one exemplary method, NC uptake into human dissociated stem cell derived β-cells was tested by incubating Cy5 loaded NCs coated with HA-Ex4 with sBCs for 24 hours at 0.5 mL of NC per 5 mL of media o (FIG. 19). NC uptake was measured by flow cytometry, where insulin positive cells were identified by GFP expression driven by the insulin promoter in sBC. It was found that NCs were taken up into ˜48% of insulin expressing sBC (FIG. 19). These results further confirm the results in dissociated human islets discussed above.

### Example 5

### NCs are Nontoxic and can Selectively Target Cells In Vitro

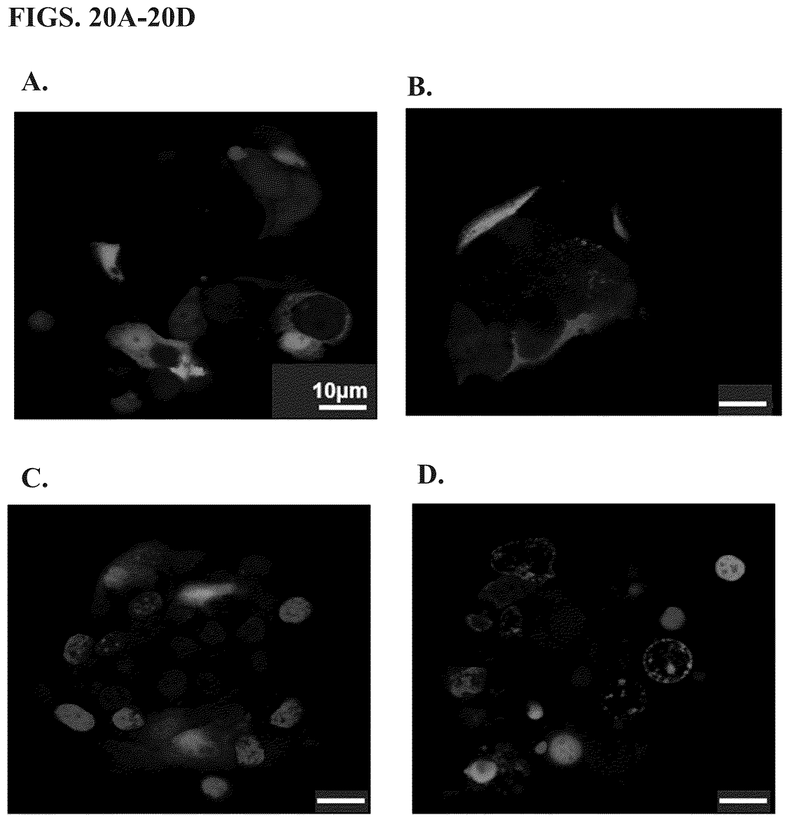

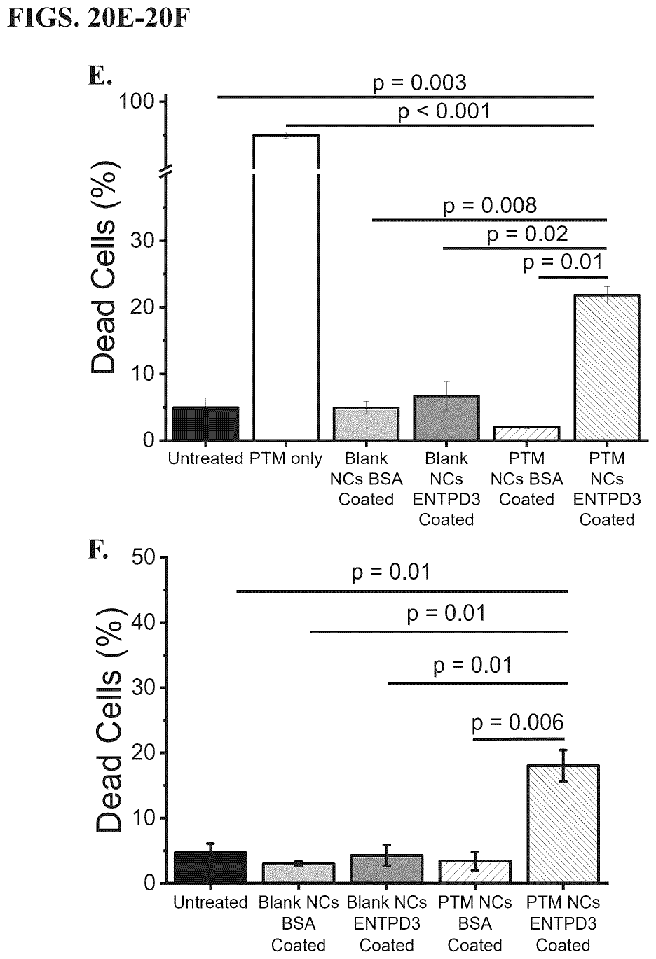

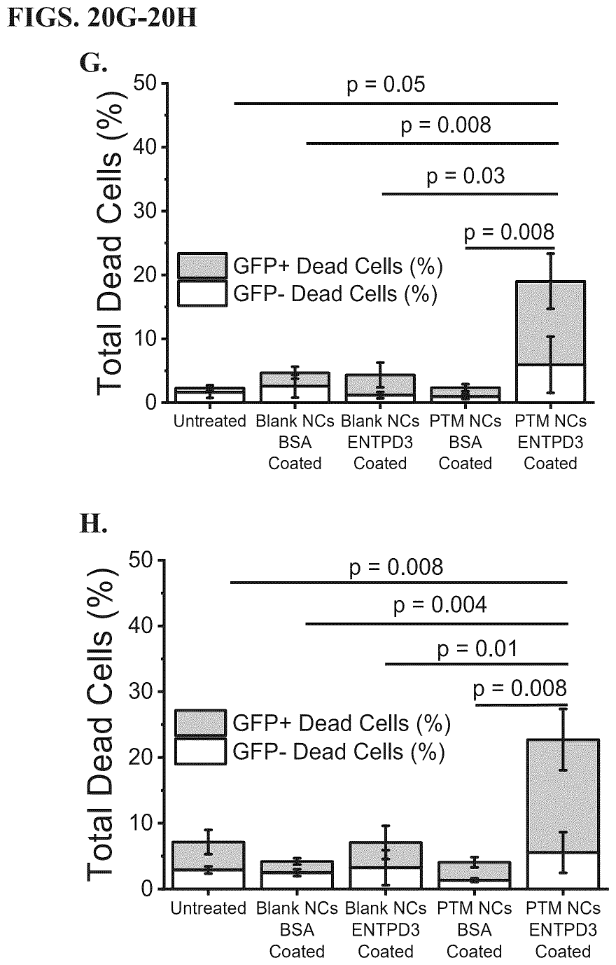

In another exemplary method, to determine whether NCs could be taken up by insulin-producing sBC clusters, a fluorescently tagged ENTPD3 antibody was adhered to the outside of blank NCs which were incubated with GFP-tagged sBCs for 24 hr. GFP expression is driven by the insulin promoter highlighting the insulin producing sBCs. By 24 hr the ENTPD3 coated NCs have been endocytosed as illustrated by the red fluorescence compared to the untreated control (FIGS. 20A-20B). This also demonstrated that the ENTPD3 antibody can be adhered to the positively charged BKC-PCL-NCs suggesting other antibodies could also be adhered and tested for selective cell targeting.

After demonstrating ENTPD3 coated NCs could be taken up by sBC clusters, in another exemplary method, it was assessed if these NCs could selectively target and deliver cargo of interest to sBC clusters. Blank or pentamidine (PTM) (B-cell toxin) loaded NCs were left uncoated or coated with either an ENTPD3 antibody or BSA for up to about 168 hours. Untreated and 1 μM free PTM were utilized as additional controls. Within 24 hours, the PTM loaded ENTPD3 coated NCs had a significantly higher percentage of dead cells compared to the untreated control (p=0.003, FIGS. 20C-20E) validating accuracy of the targeted NCs. As suspected, the positive control 1 μM free PTM treatment killed the majority of sBCs within 24 hours (FIG. 20E). A significant increase in cell death was observed with the PTM loaded ENTPD3 coated NCs compared to the blank BSA coated (p=0.008), blank ENTPD3 coated (p=0.02), and BSA coated PTM loaded NCs (p=0.01, FIG. 20E). Significant cell death was observed at 72 hours when comparing the PTM loaded ENTPD3 coated NCs to the untreated control (p=0.05), blank BSA coated (p=0.008), blank ENTPD3 coated (p=0.03), and BSA coated PTM loaded NCs (p=0.008, FIG. 20F). Although benzalkonium chloride (BKC) can be used for biological applications, it can be toxic to cells at high concentrations. Except for the ENTPD3 coated PTM loaded treatment, there was no significant change in sBC viability between the untreated sBCs to all other NC treatments (FIGS. 20E-20F) in support of the observation that the NCs are nontoxic and the concentration of BKC on the NCs is below the cytotoxic threshold making them a viable option for therapeutic agent targeted delivery.

In further exemplary methods, the number of GFP positive and GFP negative dead cells were quantified as GFP correlates to insulin positive cells. For the dead sBC clusters, 76% at 24 hours (FIG. 20G) and 69% at 72 hours were GFP positive (FIG. 20H). At 24 hours there was a significant increase in GFP positive dead cells with PTM loaded ENTPD3 coated NCs compared to the untreated (p=0.008), blank BSA coated (p=0.004), blank ENTPD3 coated (p=0.01), and BSA coated PTM loaded NCs (p=0.008, FIG. 20G). The increase in percentage of GFP positive dead cells was also significant at 72 hours when comparing the PTM loaded ENTPD3 coated NCs to the untreated (p=0.05), blank (cargo free) BSA coated (p=0.008), blank ENTPD3 coated (p=0.03), and BSA coated PTM loaded NCs (p=0.008, FIG. 20H). Collectively, this data indicates that BKC-PCL-NCs can be designed to target and deliver cargo to cells of interest and have the potential to be implemented for drug delivery applications that require selective cell targeting.

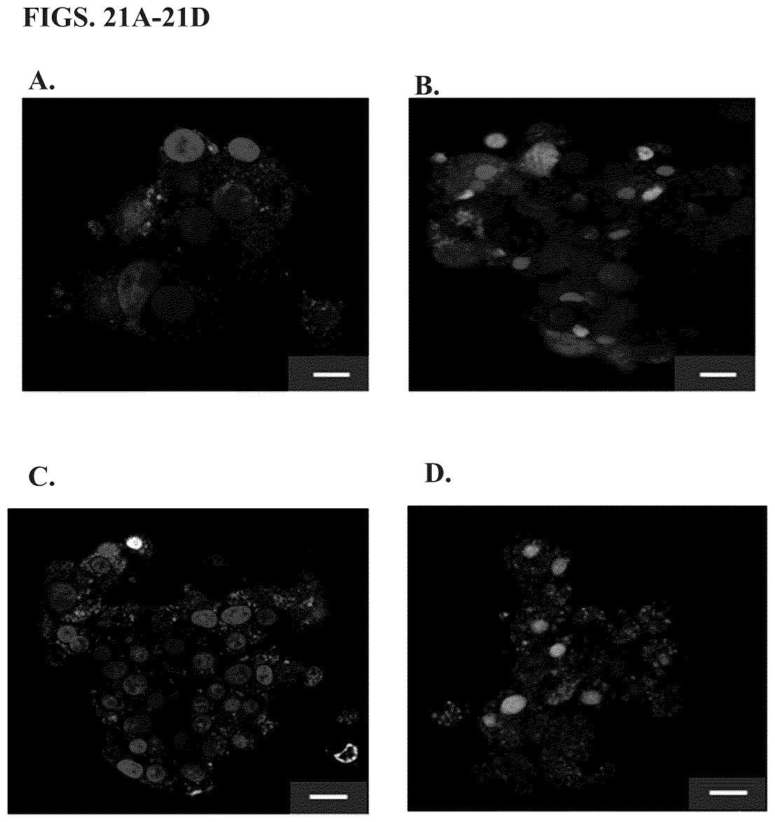

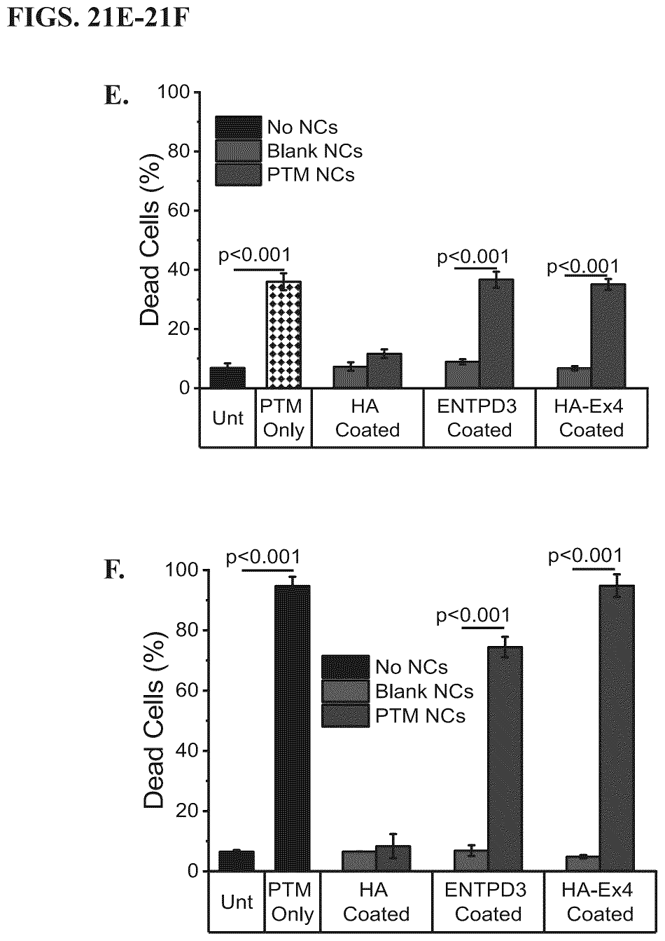

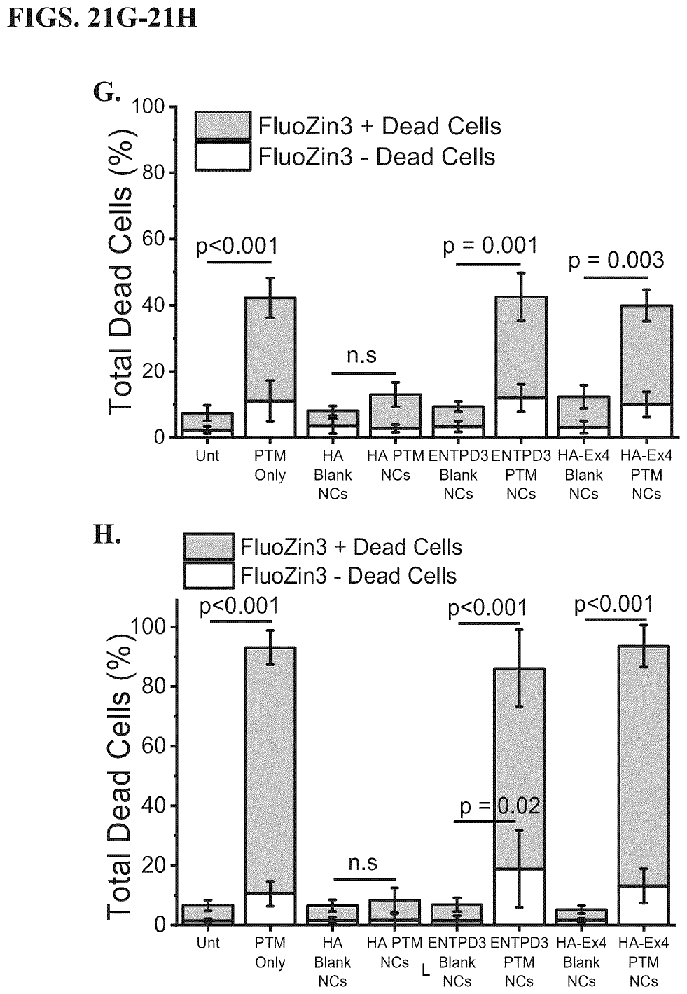

In other exemplary methods, similar experiments were performed in intact human cadaveric islets to determine if NCs could selectively target and deliver cargo of interest as in sBC. Human islets were either untreated or treated with Cy5 loaded HA-Ex4 coated NCs for 24 h and significant accumulation of NC was observed in NC treated islets (FIGS. 21A-B). Human islet β-cells were identified with FluoZin3, a zinc sensor that specifically labels β-cells (FIGS. 21A-21D). Blank (cargo free) or PTM (B-cell toxin) loaded NCs were left uncoated or coated with either an ENTPD3 antibody, HA-Ex4, or BSA for up to 168 hours. Untreated and 1 μM free PTM were utilized as additional controls. PTM induced β-cell death was measured using a live and dead cell stain with the Fluozin3 β-cell marker demonstrated in untreated human islets and islets treated with PTM loaded HA-Ex4 coated NC for 72 h (FIGS. 21C-21D). Similar results were seen in human islets as with sBC, where cell death was greatest in islets treated with NCs with either targeting peptide compared to uncoated NC at both 24 and 72 hours (FIGS. 21E-21F). Additionally, at 24 hours and 72 hours there was a significant increase in dead β-cells identified with FluoZin3 with PTM loaded ENTPD3 coated and HA-Ex4 coated NCs compared to the (FIGS. 21G-21H). Collectively, this data indicates that BKC-PCL-NCs can be designed to target and deliver cargo to the cells of interest and have the potential to be implemented for drug delivery applications that require selective human or animal cell targeting.

### Example 6

### HA-Ex4 NCs Enrich in Pancreatic Islets In Vivo

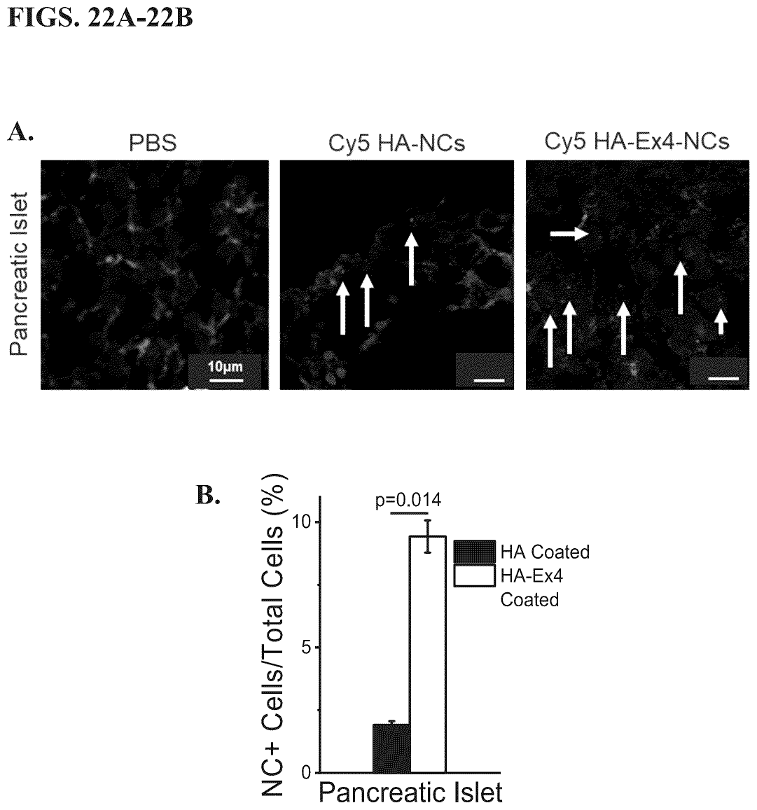

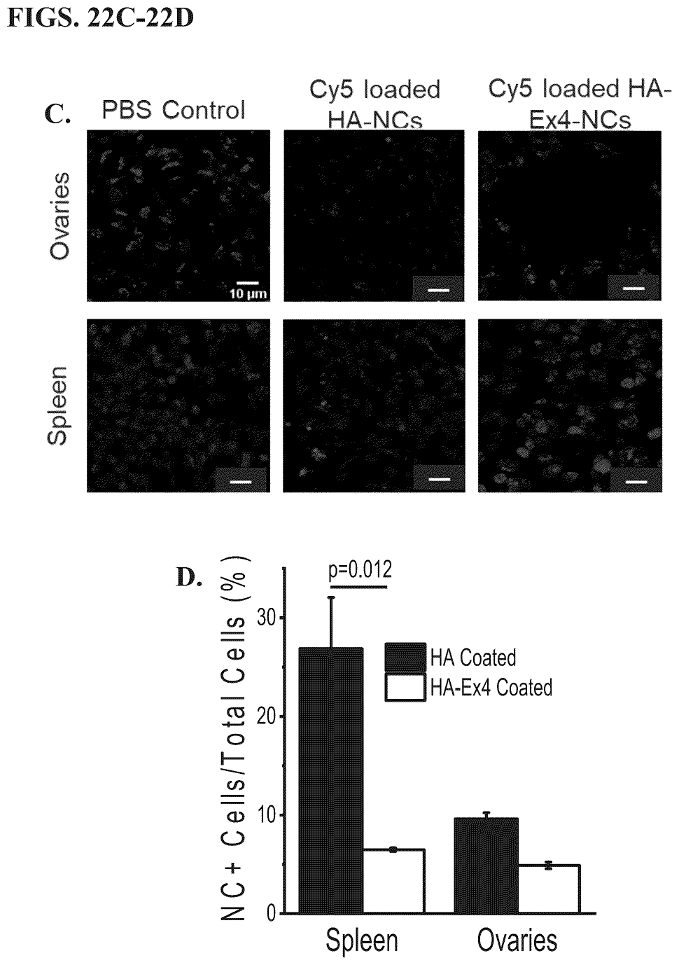

During Type 1 diabetes (TID) progression, the immune system of the subject seeks and destroys insulin producing β-cells which reside in pancreatic islets. GLP-1R is heavily expressed on the outside of pancreatic β-cells and plays important roles in maintaining glucose homeostasis. GLP-1 agonist Extendin 4 has been utilized extensively for quantifying and visualizing β-cell mass as well as for diabetes therapies. In another exemplary method, to test if Ex4 coated NCs disclosed herein could target pancreatic islets, Cy5 loaded HA coated NCS, or Cy5 loaded HA-Ex4 coated NCs were injected into the tail veins of NOD-scid mice and then sacrificed 24 hours post injection. The staining of fixed pancreatic sections revealed an enrichment of NCs in pancreatic islets compared to the PBS control (FIGS. 22A-22C). Compared to the HA coated NCs, the HA-Ex4 coated NCs were significantly enriched in pancreatic islets (p=0.014, FIG. 22B). Furthermore, most of the HA coated NCs were on the periphery of the islets compared to the HA-Ex4 coated NCs where many were further in the islet vasculature (FIGS. 22B-22C). No physical abnormalities were observed in mice injected with either the HA- or HA-Ex4 coated NCs.

Additionally, in further exemplary methods, NC uptake was quantified in the spleen, kidney, liver, and ovaries 24-hour post NC infusion. No uptake of NCs was observed in the kidney or liver for any of the tested conditions (data not shown). While some non-specific uptake of NCs was observed in mice treated with HA-coated NCs in the spleen and ovaries, non-specific uptake of NCs was significantly reduced with HA-Ex4 coated NCs (FIGS. 22C-22D), suggesting that the addition of a targeting peptide coating on the NCs reduces off-target accumulation of NC.

Together these data show the potential for using exemplary NCs provided herein to target pancreatic β-cells in vivo.

### Example 7

### Delivery of Cargo Via NCs to Improve Human β-Cell Survival and Function In Vivo

In further exemplary methods nanocapsules described herein will be formed to encapsulate δV1-1 using the W/O/W technique for hydrophilic cargo (illustrated herein in FIG. 4 to protect against cytokine toxicity in vitro) or another suitable cargo and deliver to cells or tissue in vivo. For example, the following illustrative experiment is provided to assess the ability of NCs containing δV1-1 to improve human β-cell survival and function in vivo. Three groups of sBCs ((i) unmodified, (ii) constitutively expressing PD-L1 (cP) and (iii) global HLA class I knock out (BKO), will be transplanted under the kidney capsule of certain mice (e.g., NSG-HLA-A2 mice). Of note, all sBC employed in this study will express a constitutive firefly luciferase gene allowing convenient, noninvasive quantification of total cell mass repeatedly. Differentiated sBC clusters from each genotype will be subject to detailed phenotypical analysis in vitro before transplant as described above. sBC graft viability will be monitored by luciferase bioluminescence following IP injection of luciferin substrate, measured with an IVIS Spectrum intravital imager on days 0, 3, 7, 14 and 21. BG will be monitored every 3-4 days and an intraperitoneal glucose tolerance test (IP-GTT) will be performed on day 21 to establish a baseline for graft function. On day 21 mice will receive adoptive transfer of 5e6 NOD-HLA-A2 CD8+ T cells by tail vein injection with or without addition of ENTPD3 NCs containing δV1-1. Grafts will then be again imaged on days 0, 3, 7, 14, and 21 post-transfer and BG monitored every 2-3 days. IP-GTT will be performed on days 7, 14 and 21 post-transfers to evaluate sBC function, thereafter mice will be sacrificed for graft analysis. sBC grafts will be explanted and prepared for detailed IHC of frozen sections using previously described markers.

It is expected that unmodified sBCs will be readily killed by diabetogenic T-cells within approximately 1 week. It is predicted that treatment with δV1-1 loaded NCs will delay but may not prevent graft loss of unmodified sBCs. However, in the combined setting of sBC overexpressing PD-L1 and δV1-1 loaded NCs significantly improved initial and long-term survival will likely be observed. Further, in some exemplary methods, 8V1-1 can mitigate ischemia induced cell stress and death during the engraftment phase that will reduce and likely prevent autoimmune pancreatic islet destruction and provide distinct functional improvements of cellular engraftment long term. Control sBC grafts without HLA class 1 expression will not be recognized by T-cells and serve as positive controls for these experiments.

### Example 8

### Delivery of Chemotoxic or Other Anti-Tumor Agents to Tumors In Vivo

In this example, polymer-coated NCs synthesized as demonstrated in Example 1 above can be loaded with at least one chemotoxic agent and coated with targeting peptides (e.g., folate targeting of the folate receptor in tumors in the lung, growth factors that target growth factor receptors in breast cancer tumors, including, but not limited to, EGFR, HER2, HER3, or HER4) that will direct loaded polymer-coated NCs to solid tumors via tumor-vascular factors, ligands, or other cell surface markers specific to the target solid tumor. Polymer-coated NCs are then engulfed by tumor cells of the solid tumor and the encapsulated chemotoxic agents are released only in the targeted solid tumor cells leading to specific tumor cell death and reduced off-targeting effects from administration of chemotoxic agents and reduced exposure to the chemotoxic agents.

### Example 9

Delivery of Anti-Inflammatory and/or Immunomodulatory Agents to Immune Cells In Vivo

In this example, polymer-coated NCs synthesized as demonstrated in Example 1 above are loaded with at least one anti-inflammatory and/or immunomodulatory agent and are coated with targeting peptides or molecules (e.g., CD3 targeting of T-cells, CSF1R targeting of macrophages or other surface ligand targeting of immune cells, for example) to direct the loaded polymer-coated NC to be taken up by immune cells. Then the encapsulated at least one anti-inflammatory and/or immunomodulatory agents are released over a prolonged period of time to reduce chronic or acute inflammation and the associated side effects. Inflammation conditions in a subject can be treated with a single dose of loaded polymer-coated NCs or multiple repeated doses of loaded polymer-coated NCs as determined necessary by a health professional

All of the compositions and methods disclosed and claimed herein can be made and executed without undue experimentation in light of the present disclosure. While the compositions and methods have been described in terms of embodiments, it is apparent to those of skill in the art that variations maybe applied to the compositions and methods and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

What is claimed is:

1. A nanocapsule comprising:
an inner core comprising a composition, the composition comprising at least one liquid or aqueous fatty acid and at least one therapeutic agent;
a shell coating the inner core with at least one coating layer, the coating layer comprising at least one biodegradable polymer to make a biodegradable polymer shell coating wherein the at least one coating layer of the at least one biodegradable polymer shell coating further comprises at least one positively charged surfactant on the surface of the at least one biodegradable polymer shell coating layer(s); and
at least one targeting agent associated with at least one of the at least one positively charged surfactant for targeting the nanocapsule to at least one cell, tissue, or organ in a subject.

2. The nanocapsule according to claim 1, wherein the at least one biodegradable polymer comprises at least one of polycaprolactone (PCL), poly(lactic-co-glycolic acid) (PLGA), polylactic acid (PLA), chitosan, gelatin, and a biodegradable polymer monomer size ranging from about 0.1 kilodaltons (kDa) to about 80 kDa.

3. The nanocapsule according to claim 1, wherein the at least one positively charged surfactant on the surface of the at least one biodegradable polymer shell coating layer further comprises at least one negatively charged agent, wherein the at least one negatively charged agent associates with, or binds to, the at least one positively charged surfactant and the at least one targeting agent.

4. The nanocapsule according to claim 1, wherein the at least one liquid or aqueous fatty acid composition comprises at least one liquid or aqueous lipid composition.

5. The nanocapsule according to claim 1, wherein the at least one liquid fatty acid comprises a naturally-occurring biocompatible liquid fatty acid comprising at least one of an unsaturated or a mix of saturated and unsaturated fatty acid chains wherein the naturally-occurring biocompatible liquid fatty acid is a liquid at room temperature or about 20° C. to about 25° C.

6. The nanocapsule according to claim 1, wherein the at least one liquid fatty acid comprises at least one of coconut oil, sunflower oil, vegetable oil, soybean oil, colza oil, peanut oil, mineral oil, corn oil, olive oil, palm oil, cottonseed oil, castor oil, linseed oil, borage oil, evening primrose oil, marine oils, fish oils, algae oils, oils derived from petroleum, liquid paraffin, short-chain fatty alcohols, medium-chain aliphatic branched fatty alcohols, fatty acid esters with short-chain alcohols, isopropyl myristate, isopropyl palmitate, medium-chain triglycerides, capric and caprylic triglycerides, and mixtures thereof.

7. The nanocapsule according to claim 1, wherein the at least one targeting agent comprises at least one of a polypeptide, a polynucleotide, a protein, a chimeric molecule, a glycoprotein, a whole organism, a whole cell, a pathogen, a toxin, a polysaccharide, a small molecule or metabolite, a pharmaceutical agent, or agent classified as a chemical or biological agent or other agent able to track to and bind to a biological target in a subject or a combination thereof.

8. The nanocapsule according to claim 1, wherein the at least one targeting agent comprises an antibody, a ligand, a receptor, an enzyme, a viral antigen, a bacterial antigen, a fungal antigen, a toxin, a recombinant peptide, a recombinant protein, a polypeptide derived from a target protein or pathogen, a synthetic peptide or protein, a polynucleotide derived from a target protein or pathogen, a virus-like particle, a live virus, a live, attenuated virus, an inactivated virus or a combination thereof.

9. The nanocapsule according to claim 1, wherein the at least one targeting agent comprises at least one small molecule or metabolite and the at least one small molecule or metabolite comprises a low molecular weight organic compound.

10. The nanocapsule according to claim 3, wherein the at least one negatively charged agent comprises at least one of hyaluronic acid, aggrecan, versican, syndecan, nidogen, decorin, biglycan, chondroitin sulfate, keratin sulfate, γ-polyglutamic acid, oligoethylene glycol, or other negatively charged protein or other brush polymer.

11. The nanocapsule according to claim 1, wherein the at least one therapeutic agent comprises at least one of cell proliferation agents, cell-cycle regulating agents, antisense agents, antiacids, agents against peptic ulcers and gastroesophageal reflux disease, antispasmodics, analgesics, anticholinergic drugs, propulsive drugs, antiemetics, antinausea drugs, agents for biliary therapy, agents for hepatic therapy, lipotropic agents, laxatives, anti-diarrhetic agents, intestinal adsorbents, anti-propulsive agents, anti-inflammatory drugs, agents against obesity, enzymes, hypoglycemic drugs, insulin and analogues thereof, vitamins, anabolic steroids, antithrombotic agents, antifibrinolytics, haemostatic agents, antiarrhythmic agents, cardiac stimulants, cardiac glycosides, vasodilators, antiadrenergic agents, antihypertensive drugs, diuretics, potassium-saving agents, anti-hemorrhoidals, capillary stabilizing agents, agents which act on the renin-angiotensin system, beta-blockers, selective calcium-channel blockers, non-selective calcium-channel blockers, angiotensin-converting-enzyme inhibitors, angiotensin II inhibitors, antihistamines, anesthetics, chemotherapeutic agents, anti-immune agents, corticosteroids, antiseptics, anti-acne agents, products for gynecological use, oxytocic agents, androgen, estrogen, estradiol, progestogen, progesterone, ovulation stimulants, gonadotropins, antiandrogens, drugs used in benign prostatic hypertrophy, hormones, hormone antagonists, antibiotics, antivirals, immune serum, immunoglobulins, antineoplastic agents, immunomodulatory agents, alkylation agents, antimetabolites, plant alkaloids and other natural products, cytotoxic antibiotics, immunosuppressive agents, agents for treating disorders of the musculoskeletal system, antirheumatics, muscle relaxant agents, agents which affect bone structure and mineralization, neurological agents, opioids, anti-migraine agents, anti-convulsant agents, anticholinergic agents, dopaminergic agents, antipsychotics, anxiolytics, hypnotics, sedatives, antidepressants, psychostimulants, anti-dementia agents, parasympathomimetic agents, anti-addictive disorder agents, anti-vertigo agents, antiparasitic agents, ophthalmic active ingredients, ontological active ingredients, anti-glaucoma drugs, miotics, mydriatics, cycloplegics, anti-inflammatory agents, and combinations thereof.

12. The nanocapsule according to claim 1, wherein the at least one positively charged surfactant comprises at least one of benzalkonium chloride (BKC), quaternary ammonium salts, such as cetyl trimethyl ammonium bromide, lauryl trimethyl ammonium chloride, benzyl dimethyl hexadecyl ammonium chloride, distearyl dimethyl ammonium chloride, dilauryl dimethyl ammonium chloride, dimyristyl dimethyl ammonium chloride, cetylpyridinium chloride, benzethonium chloride, and methyl benzetonium chloride.

13. The nanocapsule according to claim 1, wherein the polymer comprises PCL and the positively charged surfactant comprises BKC.

14. The nanocapsule according to claim 13, further comprising at least one negatively charged agent that binds to the at least one positively charged surfactant and further binds to the at least one targeting agent wherein at least one of the at least one negatively charged agent comprises hyaluronic acid.

15. The nanocapsule according to claim 1, wherein the at least one therapeutic agent is at least one hydrophilic or at least one hydrophobic therapeutic agent.

16. A composition comprising a nanocapsule according to claim 1, wherein the composition further comprises an excipient.

17. The composition according to claim 16, wherein the composition comprises a pharmaceutical composition and further comprises a pharmaceutically acceptable excipient.

18. A kit comprising a nanocapsule according to claim 1; and at least one container.

19-21. (canceled)

22. A method for targeting at least one cell or tissue in a subject to treat, reduce onset or prevent a health condition in the subject comprising, administering the composition according to claim 16 to the subject and inducing a response in the subject to treat, reduce onset, or prevent the health condition.

23. A method for creating at least one polymer-coated nanocapsule according to claim 1 comprising obtaining at least one biodegradable polymer, at least one fatty acid, at least one therapeutic agent, at least one targeting agent and at least one cationic surfactant and using a single emulsion procedure for hydrophobic therapeutic agents or a double emulsion procedure for hydrophilic therapeutic agents or a combined single and double emulsion for mixed hydrophobic and hydrophilic therapeutic agents; and producing targeted therapeutic agent-containing polymer-coated nanocapsules.

24-28. (canceled)
